(19) United States
(12) Patent Application Publication
(10) Pub. No.: US 2022/0228169 A1
(43) Pub. Date: Jul. 21, 2022

(54) SMALL RUMINANT LENTIVIRUS VECTOR

(71) Applicant: MOREDUN RESEARCH INSTITUTE, Midlothian (GB)

(72) Inventors: David John Griffiths, Midlothian (GB); Rebecca Kathryn McLean, Midlothian (GB)

(21) Appl. No.: 17/608,031

(22) PCT Filed: May 1, 2020
(86) PCT No.: PCT/EP2020/062198

§ 371 Date: Nov. 1, 2021

(30) Foreign Application Priority Data
May 3, 2019 (GB) 1906283.5

(51) Int. Cl. C12N 15/86; A61K 39/21

(52) U.S. Cl. CPC C12N 15/86; A61K 2039/5256; A61K 39/21

(57) ABSTRACT

The disclosure provides a plasmid and a lentiviral vector system which may be used to generate lentiviral vector particles for use in transduction. The disclosed vector system and/or plasmid improves the transduction efficiency of the generated lentiviral vector particles and may be exploited to provide compositions for raising immune responses in animals and as vaccines.

### FIELD OF THE INVENTION

The present invention provides a plasmid and a lentiviral vector system comprising said plasmid. The invention further relates to a method of using the same for generating lentiviral vector particles. The invention further relates to lentiviral vector particles and the use of lentiviral vector particles in raising an immune response in an animal or in transducing a host cell.

### BACKGROUND OF THE INVENTION

Lentiviral vectors are highly efficient tools for mediating gene transfer in animal cells and have a broad variety of applications in biomedical research and biomedicine¹. An important property of lentiviral vectors is their ability to drive sustained gene expression in a variety of dividing and non-dividing cell types. The most widely-used lentiviral vectors are derived from human immunodeficiency virus type-1 (HIV-1)^{2,3} but vector systems have also been developed from a number of non-human lentiviruses⁴, including simian immunodeficiency virus (SIV)⁵, equine infectious anaemia virus (EIAV)⁶′⁷, feline immunodeficiency virus (FIV)⁸ and bovine immunodeficiency virus^{9,10}. These lentiviral vectors can all be produced at high titres through transient transfection and ultracentrifugation. All of the non-human lentiviral vectors can efficiently infect non-dividing cells and at least one is in development for clinical use in humans^{8,11}.

Due to the pathogenicity of HIV-1, a number of safety features have been introduced into the vectors to reduce their potential harmful effects. These include the separation of viral sequences on to multiple plasmids to minimize the risk of generating replication-competent viruses through homologous recombination¹, the deletion of viral promoter/enhancer elements to minimize vector expression in recipient cells (self-inactivating (SIN) vectors)^{12,13} and the creation of integration-defective vectors to reduce the potential for insertional mutagenesis^{1,14}. These advances have decreased the potential hazards associated with HIV-1 vectors to the extent that they have been successfully used in a number of clinical therapies in humans¹⁵⁻¹⁷.

While efficient vector systems have been derived from most lentiviruses, notable exceptions are vectors derived from the small ruminant lentiviruses (SRLV), namely visna/maedi virus (VMV) and caprine arthritis encephalitis virus (CAEV)^{4,18}. Previous attempts to create lentiviral vectors from these viruses found that stable gene transfer is achievable but at an efficiency more than 100-fold lower than vectors derived from HIV-1. In one study, Berkowitz and colleagues¹⁹ reported that the low infectivity of VMV vectors in cell lines was due to cellular blocks to infection acting during reverse transcription and/or integration, although the specific mechanism(s) involved were not characterized. Similarly, vectors derived from CAEV have also been described but titres were also found to be poor compared to other lentiviral vector systems^{20,21}. The reasons underlying the reduced infectivity of SRLV vectors compared to other lentiviral systems are not well defined.

### SUMMARY OF THE INVENTION

The present disclosure provides a plasmid, which may be used to generate lentiviral vector particles for use in transduction. Also provided is a lentiviral vector system comprising the plasmid. The disclosed vector system and/or plasmid surprisingly improves the transduction efficiency of the generated lentiviral vector particles.

The lentiviral vector particles provided by this disclosure may be exploited to provide compositions for raising immune responses in animals and as vaccines. It will be appreciated that the immune responses may be protective immune responses.

The disclosed plasmid, which may otherwise be referred to as a “first plasmid” or a “transfer plasmid” is capable of delivering a nucleic acid sequence (for example a gene sequence) to a target host cell. A nucleic acid of this type shall be referred to herein after as a “nucleic acid for transfer”

The vector system described herein may comprise a plurality of plasmids.

The vector system may comprise one, two, three or four plasmids.

One or more (for example all four) of the plasmids of the vector system comprises one or more sequence(s), for example nucleic acid sequences, which are derived from a small ruminant lentivirus (SRLV); thus, the vector system is referred to as a “lentiviral vector system”.

In the context of the present invention, the phrases “small ruminant lentivirus sequence” or “small ruminant lentivirus nucleic acid sequence” refer to a nucleic acid sequence isolated or derived from a small ruminant lentivirus and/or a nucleic acid sequence exhibiting a degree of identity or homology thereto.

Useful SRLV nucleic acid sequences may encode (or be derived from) one or more features present within a SRLV genome. For example, nucleic acid sequences for use in the vector system described herein (and therefore comprised within one or more of the plasmids of that system) may be derived from, for example, nucleic acid sequences encoding (long terminal) repeat regions, unique regions, encapsidation regions/elements, poly-A regions, polyadenylation regions, promoters/regulators and the like and/or genes, for example, viral structures, enzymes and other proteins. The one or more plasmids of the vector system may further comprise nucleic acid sequences derived from other organisms—including other viruses.

As used herein, the term “degree of homology” or “degree of identity” may encompass nucleic acid and/or amino acid sequences which exhibit at least about 20%, 30%, 40%, 50%, 60%, 65%, 70%, 75%, 80%, 85%, 90%, 91%, 92%, 93%, 94%, 95%, 96%, 97%, 98% or 99% homology or identity to a reference nucleic acid or amino acid sequence. In the context of this specification, the reference nucleic acid sequence may be a coding and/or non-coding sequence of an SRLV. For example, the reference sequence may be a coding/non-coding sequence of an SRLV promoter/gene sequence.

The degree of (or percentage) “homology” between two or more (amino acid or nucleic acid) sequences may be calculated by aligning the sequences and determining the number of aligned residues which are identical and adding this to the number of residues which are not identical but which differ by redundant nucleotide substitutions—the redundant nucleotide substitution having no effect upon the amino acid encoded by a particular codon, or conservative amino acid substitutions. The combined total is then divided by the total number of residues compared and the resulting figure is multiplied by 100—this yields the percentage homology between aligned sequences.

A degree of (or percentage) “identity” between two or more (amino acid or nucleic acid) sequences may also be determined by aligning the sequences and ascertaining the number of exact residue matches between the aligned sequences and dividing this number by the number of total residues compared—multiplying the resultant figure by 100 would yield the percentage identity between the sequences.

A SRLV nucleic acid sequence for use in the present invention may be obtained or derived from any known (or deposited) SRLV genome sequence. For example, useful SRLV nucleic acid sequences may be obtained or derived from one or more of the following reference sequences:
(i) SA-OMVV (South African ovine maedi-visna virus), NCBI accession M34193.1;
(ii) Visna virus Icelandic strain 1514, NCBI accession M60610.1;
(iii) Visna/Maedi virus strain kv1772, NCBI accession L06906.1;
(iv) Visna/maedi virus strain EV1, NCBI accession S51392.1;
(v) Caprine arthritis encephalitis virus NCBI accession M33677.1; and
(vi) Small ruminant lentivirus isolate 1150, NCBI accession MH916859.1.

As stated, a SRLV nucleic acid sequence for use in a vector system of this disclosure may comprise a sequence with anywhere between about 20% and about 100% (for example 30%, 40%, 50%, 60%, 65%, 70%, 75%, 80%, 85%, 90%, 91%, 92%, 93%, 94%, 95%, 96%, 97%, 98% or 99%) identity/homology to all or part of one or more of the reference SRLV sequences described above.

As used herein, the term “nucleic acid” refers to DNA and/or RNA. Typically, the plasmid(s) of the lentiviral vector system comprise DNA. The DNA may be derived from a SRLV. DNA may comprise or consist of complementary DNA (cDNA).

The DNA may encode RNA. RNA may comprise or consist of guide RNA (gRNA), microRNA (miRNA) and/or short hairpin RNA (shRNA).

One of skill in the art will appreciate that the term “plasmid” relates to a circular nucleic acid strand.

A “nucleic acid for transfer” may be a nucleic acid sequence, which is to be expressed in a host cell. The nucleic acid sequence may be derived from a gene sequence. The nucleic acid may encode a protein and/or RNA which the user wishes to express in the host cell. For example, the “nucleic acid for transfer” may comprise a sequence (for example a gene sequence) associated with a particular disease and which the user wishes to express in a host cell. Alternatively, the “nucleic acid for transfer” may encode a mutated form of a gene. In embodiments, the “nucleic acid for transfer” encodes a peptide, protein or an antigen. The antigen can then be expressed and used to induce an immune response in a subject. Useful antigens may include antigens derived from pathogens including, for example, parasites, bacteria, fungi, viruses, protozoa and the like.

In some embodiments, the nucleic acid for transfer encodes RNA, for example gRNA, miRNA and/or shRNA. Once expressed, the shRNA and/or miRNA can be used to reduce or silence expression of a target gene in a host. Guide (g) RNA can be used to edit a target gene in a host, for example by inserting or deleting a portion of nucleic acid of the gene, such that expression of the gene is reduced, silenced or increased, typically reduced or silenced.

In the context of the present invention, “upstream” will be understood to refer to a section of nucleic acid sequence in the 5′ direction of a plasmid, relative to the sequence described, while “downstream” will be understood to refer to a section of nucleic acid sequence in the 3′ direction of a plasmid, relative to the sequence described.

In a first aspect, the disclosure provides a plasmid comprising a nucleic acid sequence encoding a promoter, a nucleic acid sequence encoding an encapsidation element, a nucleic acid sequence encoding a rev-responsive element (RRE), a site for the insertion of a nucleic acid for transfer, a woodchuck hepatitis virus post-transcriptional regulatory element (WPRE), and at least a portion of a 5′ terminal repeat and at least a portion of a 3′ terminal repeat. The nucleic acid sequence encoding an encapsidation element and the nucleic acid sequence encoding at least a portion of a 5′ terminal repeat and at least a portion of a 3′ terminal repeat are small ruminant lentivirus nucleic acid sequences. A plasmid of this type may be referred to as a first plasmid or a transfer plasmid.

By small ruminant lentivirus sequence, this will be understood to refer to a small ruminant lentivirus nucleic acid sequence.

As the skilled person will appreciate, long terminal repeats (LTRs) are DNA sequences repeated at each end of a viral genome.

It will be appreciated that the encapsidation element is an RNA element that is recognised by a gag protein for encapsidation. The nucleic acid for transfer encoded on the first plasmid can be packaged by the gag protein, such that lentiviral vector particles produced from the plasmid(s) comprise the nucleic acid for transfer.

According to a second aspect there is provided a lentiviral vector system comprising the plasmid of the first aspect. The lentiviral vector system optionally further comprises a packaging plasmid. The packaging plasmid comprises a nucleic acid sequence encoding a gag polyprotein and a gag-pol polyprotein. The packaging plasmid may otherwise be referred to as a second plasmid. The packaging plasmid may comprise a small ruminant lentivirus sequence.

The system may further comprise an additional plasmid comprising a nucleic acid sequence encoding an envelope protein. This additional plasmid may be otherwise referred to as a third plasmid.

In embodiments, the system may comprise an additional plasmid comprising a nucleic acid sequence encoding a rev protein. This plasmid may otherwise be referred to as a fourth plasmid. The fourth plasmid may optionally comprise a small ruminant lentivirus nucleic acid sequence. The nucleic acid sequence encoding a rev protein may be derived from a small ruminant lentivirus sequence.

Surprisingly, the inventors have found that the use of such a system improves the transduction efficiency of the lentiviral vector particles produced from these plasmids.

Optional features of each of these plasmids are described herein.

According to a third aspect, there is provided a lentiviral vector particle derived from a small ruminant lentivirus, wherein the lentiviral vector particle comprises a nucleic acid sequence encoding an encapsidation element, a nucleic acid sequence encoding a rev-responsive element (RRE), a nucleic acid sequence for transfer, a woodchuck hepatitis virus post-transcriptional regulatory element (WPRE), and at least a portion of a 5′ terminal repeat and at least a portion of a 3′ terminal repeat. The nucleic acid sequence encoding an encapsidation element and the nucleic acid sequence encoding at least a portion of a 5′ terminal repeat and at least a portion of a 3′ terminal repeat are small ruminant lentivirus nucleic acid sequences.

The nucleic acid of the lentiviral vector particle(s) is preferably RNA.

In some embodiments, the nucleic acid sequence encoding the rev-responsive element (RRE) is a small ruminant lentivirus sequence.

A plurality of lentiviral vector particles may be provided.

A portion of a 5′ long terminal repeat may comprise at least one of at least a portion of an U3 region, an R region and a U5 region. A portion of a 3′ long terminal repeat may comprise at least one of a U3 region, an R region, and a U5 region. In embodiments, the lentiviral vector particle comprises a 5′ R region and a 5′U5 region. The lentiviral vector particle may comprise a 5′ R region, a 5′ U5 region and a 5′ U3 region. In embodiments, the lentiviral vector particle comprises a 3′ R region and a 3′ U5 region. In embodiments, the lentiviral vector particle comprises a 3′ R region, a 3′ U5 region and a 3′U3 region.

In embodiments, the lentiviral vector particle comprises a 5′ long terminal repeat. In some embodiments, the lentiviral vector particle comprises a 3′ long terminal repeat. The lentiviral vector particle may comprise a 5′ long terminal repeat and a 3′ long terminal repeat.

In embodiments a portion of the 3′ long terminal repeat, for example a portion of the 3′ U3 region is absent from the lentiviral vector particle. The absence of a portion of the 3′ long terminal repeat may introduce deletions into the 5′ long terminal repeat during reverse transcription. This deletes the viral promoter in transduced cells and prevents further transduction, hence generating self-inactivating lentiviral vector particles. Thus, in some embodiments the lentiviral vector particle is self-inactivating.

In embodiments, the 3′ long terminal repeat or a portion thereof of the lentiviral vector particle does not contain a TATA box nucleic acid sequence.

The lentiviral vector particle may comprise a plurality of nucleic acid sequence(s) for transfer. Where the lentiviral vector particle comprises a plurality of nucleic acid sequences for transfer, the nucleic acid sequences for transfer may be operatively linked, for example using a nucleic acid linker sequence encoding an IRES or 2A peptide cleavage signal. As the skilled person will appreciate, operative linkage enables co-expression of two or more genes for transfer and are standard in the art.

The lentiviral vector particle may comprise a rev protein. The rev protein may be derived from a small ruminant lentivirus.

The lentiviral vector particle may comprise a gag polyprotein and a gag-pol polyprotein. As used herein, polyprotein refers to a plurality of proteins. The polyproteins are cleaved during replication to yield mature proteins. For gag, these are matrix, capsid and nucleocapsid proteins. For gag-pol, these are matrix, capsid, nucleocapsid, protease, reverse transcriptase, dUTPase and an integrase enzyme.

The lentiviral vector particle may comprise an integrase enzyme which is non-functional or not complete. In such embodiments, it will be understood that lentiviral vector particle will be integration-defective. Integration-defective will be understood to mean that the DNA of lentiviral vector particles does not integrate into the genome of the host cell following transduction. Advantageously, this reduces the risk of replication-competent recombinant viruses. This improves safety to the user and/or to any subject administered the lentiviral vector particle. Integration-defective lentiviral vector particles also reduce the risk of causing insertional mutagenesis to the genome of the host cell from the lentiviral vector particle.

In embodiments where the integrase enzyme is non-functional, the integrase enzyme may comprise one or more mutations to alanine, valine and/or glycine in the integrase enzyme. In embodiments, the one or more mutations comprise a mutation at position E154, D66 and/or D118 in the integrase enzyme. By “a mutation”, this may be understood to refer to an amino acid different to the wild type amino acid at a particular position. In embodiments, the one or more mutations are selected from E154A, E154V, E154G and/or D66A, D66V, D66G and/or D118A, D118V, D118G.

The lentiviral vector particle may comprise an envelope protein, which optionally forms an envelope for the lentiviral vector particle. In embodiments, the envelope protein is derived from a vesicular stomatitis virus (VSG). Other suitable envelope proteins are well known in the field. These include, but are not limited to, the SRLV Env protein, baculovirus gp64, and other viral glycoproteins

It will be appreciated that the lentiviral vector particle(s) may further comprise a nucleic acid sequence encoding a PPT (polypurine tract). The lentiviral vector particle(s) may optionally further comprise a nucleic acid sequence encoding a cPPT/cts (central polypurine tract and central termination sequence).

In embodiments, the lentiviral vector particle may comprise or further comprise a nucleic acid sequence encoding a reporter gene.

It will be appreciated that the lentiviral vector particles are for the transfer of a nucleic acid into a host cell. Said nucleic acid being the nucleic acid for transfer comprised within the first/transfer plasmid.

According to a fourth aspect, the disclosure provides a method of generating lentiviral vector particles derived from a small ruminant lentivirus, the method comprising transfecting a cell with the plasmid or lentiviral vector system according to the first or second aspect.

According to a fifth aspect, the disclosure provides a lentiviral vector particle derived from a small ruminant lentivirus producible according to the method of the fourth aspect.

According to a sixth aspect, the invention provides a lentiviral vector particle derived from a small ruminant lentivirus according to the third or the fifth aspect for use in transducing a host cell.

According to a seventh aspect there is provided use of the lentiviral vector particle derived from a small ruminant lentivirus according to the third or the fifth aspect for transducing a host cell.

The host cell may be a eukaryotic cell such as, for example a plant, insect, fish, protozoal, nematode, ectoparasite, mammalian, and/or fungal cell. In some embodiments, the host cell is a mammalian cell, for example an ovine cell. The ovine cell may comprise or consist of an ovine immune cell such as a dendritic cell, e.g. a monocyte-derived dendritic cell or a macrophage, e.g., monocyte-derived macrophages. A plurality of host cells may be transduced.

According to an eighth aspect, there is provided a lentiviral vector particle derived from a small ruminant lentivirus according to the third or the fifth aspect for use in raising an immune response in an animal.

The animal may be any mammalian subject, for example a dog, cat, rat, mouse, human, sheep, goat, donkey, horse, cow, pig and/or chicken.

In embodiments, the animal is an ovine animal, a caprine animal, an equine animal, a porcine animal, a bovine animal or a human. In embodiments, the animal is an ovine animal. By “ovine animal”, this will be understood to include sheep.

The skilled person will appreciate that the term “caprine” includes goats, while “bovine” includes cattle. Equine is a term that will be understood to include horses. As used herein, the term “porcine” includes pigs.

An immune response which contributes to an animal's ability to resolve an infection/infestation and/or which helps reduce the symptoms associated with an infection/infestation may be a referred to as a “protective response”. In the context of this invention, the immune responses raised through exploitation of the lentiviral vector particle(s) described herein may be referred to as “protective” immune responses. The term “protective” immune response may embrace any immune response which: (i) facilitates or effects a reduction in host pathogen burden; (ii) reduces one or more of the effects or symptoms of an infection/infestation; and/or (iii) prevents, reduces or limits the occurrence of further (subsequent/secondary) infections.

Thus, a protective immune response may prevent an animal from becoming infected/infested with a particular pathogen and/or from developing a particular disease or condition.

An “immune response” may be regarded as any response which elicits antibody (for example IgA, IgM and/or IgG or any other relevant isotype) responses and/or cytokine or cell mediated immune responses. The immune response may be targeted to the product of the nucleic acid for transfer. For example, where the nucleic acid for transfer encodes a protein (for example an antigen), the immune response may comprise antibodies which have affinity for epitopes of the protein (or antigen).

The invention further provides as a ninth aspect an immunogenic composition or vaccine comprising the lentiviral vector particle(s) according to the third or the fifth aspect.

In an tenth aspect, there is provided a kit or composition comprising the plasmid of the first aspect of the lentiviral vector system of the second aspect. By way of example, a composition or kit may comprise:
(i) (a quantity of) a first (transfer) plasmid as described herein; and optionally;
(ii) (a quantity of) a second (packaging) plasmid as described herein.

The composition or kit may further comprise (a quantity of) a third and/or fourth plasmid as described herein.

As used herein, transfecting refers to the process of introducing free nucleic acid into a eukaryotic cell by allowing the nucleic acid to cross the plasma membrane of the eukaryotic cell. By free nucleic acid, this will be understood to refer to nucleic acid which is not contained within a virus, virus-like particle or other organism; i.e. the nucleic acid is independent of an organism (although it will be appreciated that the nucleic acid may be derived or isolated from the nucleic acid sequence of an organism).

Methods of transfection typically involve altering the plasma membrane such that free nucleic acid can cross the plasma membrane (for example, electroporation methods) or complexing the free nucleic acid with a reagent that enables the free nucleic acid to cross the plasma membrane.

Thus, in the context of the present invention, transfecting refers to the introduction of the plasmid(s) of the invention into a cell. Once introduced into the cell, the plasmid(s) is transcribed and the transcripts are translated into viral proteins. The viral proteins are packaged by the cell to form replication deficient lentiviral vector particles. The lentiviral vector particles exit the cell into a supernatant by budding from the cell membrane. Lentiviral vector particles can then be harvested from the supernatant.

Cells typically used for transfection include human embryonic kidney (HEK) 293 cells, for example HEK 293T cells. Cells typically used for transfection may be referred to in the context of the present application as packaging cells. Other suitable cells for transfection will be known to those skilled in the art.

Various transfection methods are known to those skilled in the art. Transfecting may comprise polyethylenimine, poly-L-lysine, calcium phosphate, electroporation or liposomal-based methods. In embodiments, transfecting may comprise polyethylenimine, calcium phosphate or liposomal-based methods.

It will be appreciated that a variety of liposomal-based reagents are available commercially for liposomal-based methods of transfection.

Liposomal methods may include, but may not be limited to lipofectamine-based transfection or FuGENE®HD (Promega Corporation, Wisconsin, USA)-based transfection.

Further information regarding transformation/transfection techniques may be found in Current Protocols in Molecular Biology (2019) which is incorporated herein by reference.

In a further aspect, the present invention provides host cells transfected with the plasmid of the first aspect or the lentiviral vector system of the second aspect as described herein. Eukaryotic cells, such as, for example plant, insect, fish, protozoal, nematode, ectoparasite, mammalian, and/or fungal cells, may be transfected with one or more of the plasmids of the vector system described herein. Host cells transfected with the plasmid of the first aspect or the lentiviral vector system of the second aspect as described herein may be referred to as packaging cells. In embodiments, the host cell is a mammalian cell, for example a HEK 293T cell.

In some embodiments, the packaging cells stably express the lentiviral vector particles or components of the lentiviral vector particles. By “stably express”, the expression is not transient, i.e. expression is maintained for at least 2 weeks, 1 month, 2 months, 3 months, 4 months, 5 months, 6 months, 1 year or 2 years.

The packaging cells may be from a packaging cell line. As the skilled person will appreciate, a cell line is a cell which has been immortalised such that it can continue to proliferate and so can be grown indefinitely in vitro.

Another aspect provides host cells transduced with the lentiviral vector particles derived from a small ruminant lentivirus according to the third or fifth aspects of the invention. The host cells may be eukaryotic cells, such as, for example plant, insect, fish, protozoal, nematode, ectoparasite, mammalian, and/or fungal cells. In some embodiments, the host cells are mammalian cells, for example ovine cells. The ovine cells may comprise ovine immune cells such as dendritic cells, e.g. monocyte-derived dendritic cells or macrophages, e.g., monocyte-derived macrophages.

One of skill in the art will appreciate that transduction refers to a process whereby nucleic acid is introduced into a host cell by an infective process, i.e. using one or more viral mechanisms of infection. Thus, in the context of the present invention, transduction refers to a process whereby nucleic acid is introduced into a host cell by virus-like particles, typically the lentiviral vector particles. In embodiments, transduction leads to stable integration of the nucleic acid of the lentiviral vector particles into the host cell.

The site for the insertion of a nucleic acid for transfer of the transfer plasmid of the first and second aspect may comprise a cloning site, for example a multiple cloning site. Multiple cloning sites are known in the art as a section of nucleic acid containing a plurality of restriction sites. One of skill will readily understand that nucleic acid sequences can be engineered with restriction sequences so that they can be inserted into any given site within a multiple cloning site.

In some embodiments the transfer (or first) plasmid comprises one or more (for example a plurality of) sites into which a nucleic acid sequence for transfer can be inserted.

In embodiments, the first plasmid comprises a nucleic acid for transfer inserted into the site for the insertion of a nucleic acid for transfer. The first plasmid may comprise one or more (for example a plurality of) nucleic acid sequence(s) for transfer.

Where the first (or transfer) plasmid comprises a plurality of nucleic acid sequences for transfer, one or more of these nucleic acid sequences may be in a reverse orientation relative to one or more other nucleic acid sequences for transfer. For example, while one or more nucleic acid sequences for transfer may be in the 5′ to 3′ orientation, one or more other nucleic acid sequences for transfer may be in a 3′ to 5′ orientation relative to the one or more of the other nucleic acid sequences for transfer in the plasmid.

The nucleic acid sequences for transfer may be operatively linked, for example using a nucleic acid linker sequence encoding an IRES or 2A peptide cleavage signal. As the skilled person will appreciate, operative linkage enables co-expression of two or more genes for transfer and are standard in the art.

Inclusion of the WPRE into the first plasmid surprisingly improves the transduction efficiency of the generated lentiviral vector particles. In some embodiments, the WPRE comprises SEQ ID NO:1, or a fragment or variant thereof.

By “variant” of a sequence, we include insertions, deletions and substitutions, either conservative or non-conservative. In particular, we include variants of the nucleotide sequence where such changes do not substantially alter the biological activity of the nucleic acid sequence or of the product encoded by the nucleic acid sequence. A skilled person would know that such sequences can be altered without the loss of biological activity. In particular, single changes in the nucleotide sequence may not result in an altered amino acid sequence following expression of the sequence. Furthermore, if changes in the nucleotide sequence result in the incorporation of an alternative amino acid, but wherein the physio-chemical properties of the respective amino acid(s) are not substantially changed (for example, conservative substitutions such as Gly, Ala; Val, Ile, Leu; Asp, Glu; Asn, Gln; Ser, Thr; Lys, Arg; and Phe, Tyr), the functionality of the respective protein should not be affected. Moreover, small deletions within non-functional regions of the protein can also be tolerated and hence are considered “variants” for the purpose of the present invention. The experimental procedures described herein can be readily adopted by the skilled person to determine whether a “variant” can still function.

It is preferred if the variant has a sequence which has at least 75%, yet still more preferably at least 80%, in further preference at least 85%, in still further preference at least 90% and most preferably at least 95%, 97%, 98% or 99% identity with the “naturally occurring” nucleotide sequence.

The site into which a nucleic acid sequence for transfer can be inserted (the insertion site) may be located upstream of the WPRE. The site may be adjacent to the WPRE. The term “adjacent to” will be understood to mean that the site is next to the WPRE. In embodiments, the insertion site is 5′ to the WPRE. Hence, in some embodiments the site is adjacent to the WPRE in a 5′ position.

The first (or transfer) plasmid further comprises a nucleic acid sequence encoding a promoter. In embodiments, the promoter comprises a cytomegalovirus (CMV) promoter. Other suitable promoters will be known to the skilled person. An exemplary cytomegalovirus promoter sequence is provided by SEQ ID NO:2. Hence, in some embodiments the nucleic acid sequence encoding a promoter may comprise or consist of SEQ ID NO:2, or a fragment or variant thereof.

The first plasmid may comprise a plurality of nucleic acid sequences encoding a plurality of promoters, for example two promoters. Optionally, each promoter comprises a CMV promoter. In embodiments comprising two promoters, a first promoter is upstream of the insertion site. A second promoter may also be upstream of the insertion site. In some embodiments, the second promoter is located downstream of the insertion site, optionally between the insertion site and the WPRE.

In embodiments comprising a plurality of nucleic acid sequences for transfer, the first plasmid may comprise a plurality of promoters, wherein each promoter is upstream of a nucleic acid sequence for transfer.

As the skilled person will appreciate, the 5′ long terminal repeat comprises a viral promoter. A portion of a 5′ long terminal repeat may comprise at least one of at least a portion of an U3 region, an R region and a U5 region. A portion of a 3′ long terminal repeat may comprise at least one of a U3 region, an R region, and a U5 region. In embodiments, the first plasmid comprises a 5′ R region and a 5′U5 region. The first plasmid may comprise a 5′ R region, a 5′ U5 region and a 5′ U3 region. In embodiments, the first plasmid comprises a 3′ R region and a 3′ U5 region. In embodiments, the first plasmid comprises a 3′ R region, a 3′ U5 region and a 3′ U3 region.

SEQ ID NO:3 is an exemplary sequence of a portion of a small ruminant lentivirus U3 region. SEQ ID NO:4 is an exemplary sequence of a small ruminant lentivirus U3 region. An exemplary small ruminant lentivirus R region is provided by SEQ ID NO:5, while an exemplary small ruminant lentivirus U5 region is provided by SEQ ID NO:6. The portion of the 3′ long terminal repeat may additionally or instead of comprise SEQ ID NO:7.

Thus, in some embodiments the portion of the 5′ long terminal repeat comprises at least one of SEQ ID NO:3, SEQ ID NO:4, SEQ ID NO:5 and/or SEQ ID NO:6, or a fragment or variant thereof.

In some embodiments, the portion of the 3′ long terminal repeat comprises at least one of SEQ ID NO:3, SEQ ID NO:4, SEQ ID NO:5, SEQ ID NO:6 and/or SEQ ID NO:7, or a fragment or variant thereof.

In embodiments the first plasmid comprises a 5′ long terminal repeat. In some embodiments the first plasmid comprises a 3′ long terminal repeat. The first plasmid may comprise a 5′ long terminal repeat and a 3′ long terminal repeat.

In embodiments at least a portion of the 5′ long terminal repeat is between the nucleic acid sequence encoding a promoter and the site for the insertion of a nucleic acid for transfer. In embodiments, the 5′ long terminal repeat is between the nucleic acid sequence encoding a promoter and the site for the insertion of a nucleic acid for transfer.

In embodiments comprising two promoters, the 5′ long terminal repeat, or a portion thereof, may be between the nucleic acid sequence encoding the first promoter and the site for the insertion of a nucleic acid for transfer. In such embodiments, the nucleic acid sequence encoding the first promoter or a promoter is upstream of the 5′ long terminal repeat, or a portion thereof. The positioning of the promoter upstream of the 5′ long terminal repeat, or a portion thereof, facilitates high-level expression in cells such as HEK 293T cells.

In embodiments a portion of the 3′ long terminal repeat, for example a portion of the 3′ U3 region is absent from the first plasmid. By deleting a portion of the 3′ long terminal repeat, these deletions are introduced into the 5′ long terminal repeat during reverse transcription. This deletes the viral promoter in transduced cells and prevents further transduction, hence generating self-inactivating vectors. SEQ ID NO:8 is an exemplary small ruminant lentivirus 3′ U3 region wherein a portion (147 base pairs) of its sequence is absent. SEQ ID NO:9 is another exemplary small ruminant lentivirus 3′ U3 region wherein a portion (173 base pairs) of its sequence is absent. Thus, in some embodiments the 3′ long terminal repeat of the first plasmid comprises SEQ ID NO:8 or SEQ ID NO:9, or a fragment or variant thereof. SEQ ID NO:8 or SEQ ID NO:9 may be used instead of SEQ ID NO:3 or SEQ ID NO:4.

The encapsidation element of the transfer plasmid may comprise a 5′ leader sequence, for example a small ruminant lentivirus 5′ leader sequence, such as a sequence comprising or consisting of SEQ ID NO:10, or a fragment or variant thereof.

The first (or transfer) plasmid may comprise a polypurine tract and terminal sequence enhancer element, optionally upstream of the at least a portion of the 3′ long terminal repeat or the 3′ long terminal repeat. The polypurine tract and terminal sequence enhancer element may be a small ruminant lentivirus sequence. An exemplary small ruminant lentivirus polypurine tract and terminal sequence enhancer element is SEQ ID NO:11.

An exemplary portion of a transfer plasmid may be provided by SEQ ID NO:12. The functional regions of SEQ ID NO:12 are as follows:
Nucleotides 9-612, human CMV immediate early promoter;
Nucleotides 613-1826, VMV partial U3 region (SEQ ID NO:3), R region (SEQ ID NO:5), U5 region (SEQ ID NO:6), 5′ leader region (SEQ ID NO:10) and portion of nucleic acid sequence encoding encapsidation element (SEQ ID NO:13);
Nucleotides 1846-2046, VMV RRE (SEQ ID NO:14) (represents nucleotides 7921-8121 of VMV strain KV1772 (Gen Bank accession NC_001452.1);
Nucleotides 2066-2594, putative VMV cPPT/cts (SEQ ID NO:15) (cPPT1; represents nt 4969-5500 of KV1772) mutated Nsil site is underlined;
Nucleotides 2677-3270, internal human CMV immediate early promoter (SEQ ID NO:2);
Nucleotides 3288-4052, an optional nucleic acid encoding an EGFP for transfer (SEQ ID NO:16);
Nucleotides 4057-4650, an WPRE (SEQ ID NO:1); and
Nucleotides 4687-5228, VMV 3′ region (SEQ ID NO:7), 3′ U3 region (SEQ ID NO:4), R region (SEQ ID NO:5) and U5 region (SEQ ID NO:6) (bold, KV1772 strain).

SEQ ID NO:12 comprises Not I (GCGGCCGC) and Sal I (GTCGAC) restriction sites, which, for example, can be used for cloning of the sequence into a plasmid such as a pBluescript plasmid.

In some embodiments, the encapsidation element of the transfer plasmid comprises a 5′ leader sequence and a portion of a nucleic acid sequence encoding a gag polyprotein, for example SEQ ID NOs 10 and 13, or a fragment or variant thereof.

SEQ ID NO:17 comprises SEQ ID NO:12 without the nucleic acid encoding the WPRE and the EGFP for transfer in a pBluescript backbone (SEQ ID NO:20). SEQ ID NO:17 may otherwise be referred to herein as a pCV plasmid.

Exemplary first plasmids may be provided by SEQ ID NO:18 or SEQ ID NO:19. SEQ ID NO:18 comprises SEQ ID NO:12 without the nucleic acid encoding the EGFP for transfer in a pBluescript backbone (SEQ ID NO:20). SEQ ID NO:18 may otherwise be referred to herein as a pCVW plasmid. SEQ ID NO:19 comprises SEQ ID NO:12 in a pBluescript backbone (SEQ ID NO:20). SEQ ID NO:19 may otherwise be referred to herein as a pCVW-CG plasmid.

The first plasmid may comprise SEQ ID NO:12, SEQ ID NO: 18 or SEQ ID NO:19 absent nucleotides from position 4890, position 4885, position 4880 or position 4878 to position 5000, position 5005, position 5010, position 5015, position 5020, position 5025, position 5029, position 5030, position 5035, position 5040, position 5045 or position 5047. In embodiments, the first plasmid comprises SEQ ID NO:12, SEQ ID NO:18 or SEQ ID NO:19 absent nucleotides from position 4878 to position 5029.

In embodiments, the 3′ long terminal repeat or a portion thereof of the first plasmid does not contain a TATA box nucleic acid sequence.

The first plasmid may comprise SEQ ID NO:12, SEQ ID NO:18 or SEQ ID NO:19 absent nucleotides from position 4878 to position 5047.

The first plasmid may comprise SEQ ID NO:21 (which may otherwise be referred to herein as a pCVW-SIN1 plasmid) or SEQ ID NO:22 (which may otherwise may referred to herein as a pCVW-SIN2 plasmid). SEQ ID NO:21 comprises SEQ ID NO:12 in a pBluescript plasmid backbone except that SEQ ID NO:4 is replaced by SEQ ID NO:8 and absent the nucleic acid encoding the EGFP for transfer. SEQ ID NO:22 comprises SEQ ID NO:1 in a pBluescript plasmid backbone except that SEQ ID NO:4 is replaced by SEQ ID NO:9 and absent the nucleic acid encoding the EGFP for transfer. It will be appreciated that SEQ ID NO:9 does not comprise a TATA box nucleic acid sequence. It will be appreciated that the replacement of SEQ ID NO:4 with SEQ ID NO: 8 or 9 in SEQ ID NO:21 or SEQ ID NO:22 generate self-inactivating vectors.

Optionally, the first plasmid comprises a central polypurine tract and central termination sequence (cPPT) enhancer element. In some embodiments the small ruminant lentivirus of the first plasmid comprises a cPPT enhancer element, optionally SEQ ID NO:15. In other embodiments the plasmid does not comprise a cPPT enhancer element. The inventors have found that the absence of the cPPT element does not detrimentally affect the transduction efficiency of any generated lentiviral vector particles. This is surprising because previous studies have suggested that the cPPT element improves transduction efficiency.

The first plasmid may comprise a nucleic acid sequence encoding one or more Mason-Pfizer monkey virus constitutive transport elements (CTE). In embodiments the one or more CTEs are positioned between the WPRE and the PPT. In embodiments the first plasmid comprises at least two CTEs. An exemplary nucleic acid sequence encoding two CTEs is provided by SEQ ID NO:23. The inventors believe that the CTEs may act as a framework allowing the expression of RNA from the first plasmid.

In some embodiments the nucleic acid sequence encoding the rev-responsive element (RRE) is a small ruminant lentivirus sequence, for example SEQ ID NO:14, or a fragment or variant thereof. Rev-responsive elements are known in the art and will be understood to refer to RNA sequences which act as a framework on which the Rev protein assembles. The rev-responsive element and Rev protein encoded by the fourth plasmid may facilitate the expression of RNA from the first plasmid.

The second plasmid may comprise a nucleic acid sequence encoding a small ruminant lentivirus sequence, for example a visna/maedi virus (VMV) nucleic acid sequence.

The nucleic acid encoding the gag polyprotein and gag-pol polyprotein of the second plasmid may be a small ruminant lentivirus nucleic acid sequence. The second plasmid may comprise one or more additional small ruminant lentivirus sequences.

It will be appreciated that at least a portion of the nucleic acid sequence encoding the gag polyprotein and the gag-pol polyprotein of the second plasmid comprises a nucleic acid sequence encoding an integrase enzyme. As the skilled person will appreciate, the nucleic acid encoding the gag polyprotein and the gag-pol polyprotein will be transcribed into RNA in a host cell, followed by translation of the RNA into the polyproteins, gag and gag-pol.

An exemplary small ruminant lentivirus nucleic acid sequence encoding the gag polyprotein and the gag-pol polyprotein is SEQ ID NO:24. In some embodiments, the nucleic acid sequence encoding the gag polyprotein and the gag-pol polyprotein comprises SEQ ID NO:24, or a fragment or variant thereof.

The nucleic acid sequence encoding an integrase enzyme (i.e. a portion of the nucleic acid sequence encoding the gag and gag-pol polyproteins) may not be a sequence encoding a functional or complete viral integrase. In embodiments where the nucleic acid sequence encoding an integrase is not a sequence encoding a functional or complete viral integrase, it will be understood that the vector system will be integration-defective. Integration-defective will be understood to mean that the DNA of lentiviral vector particles produced from the vector system does not integrate into the genome of the host cell following transduction. Advantageously, an integration-defective vector system reduces the risk of producing replication-competent recombinant viruses from the vector system. This improves safety to the user and/or to any subject administered the lentiviral vector particle. Integration-defective vector systems also reduce the risk of causing insertional mutagenesis to the genome of the host cell from the vector system or lentiviral vector particles.

In embodiments where the nucleic acid sequence encoding an integrase does not encode a functional integrase, the nucleic acid sequence encoding the integrase may encode one or more mutations in the integrase enzyme. The nucleic acid sequence encoding the integrase may encode one or more mutations to alanine, valine and/or glycine in the integrase enzyme. In embodiments, the one or more mutations comprise a mutation at position E154, D66 and/or D118 in the integrase encoded by the nucleic acid sequence. In embodiments the one or more mutations are selected from E154A, E154V, E154G and/or D66A, D66V, D66G and/or D118A, D118V, D118G. The nucleic acid sequence encoding the integrase may encode an E154A mutation, a D66A mutation and/or a D118A mutation in the integrase enzyme. It will be appreciated that these mutations relate to the mutations in the amino acid sequence of the integrase following transcription and translation of the nucleic acid sequence encoding the integrase. The skilled person will be aware of the nucleic acid code and thus will be aware of suitable nucleic acid changes and codons which will result in each of the above mutations.

Exemplary primers for the production of such mutants are described herein. Suitable primers for the generation of an E154A mutation may comprise GGCAAGTGGATTACACTCATTTTGAAG (SEQ ID NO:25), CCTGGCCACTAGAGCTTGAGACTGTGG (SEQ ID NO:26), GAGTGTAATCCACTTGCCAATGATCT (SEQ ID NO:27) and CAAGCTCTAGTGGCCAGGGCTCATCAG (SEQ ID NO:28).

Suitable primers for the generation of a D66A/E154A dual mutation may comprise SEQ ID NO: 26, SEQ ID NO:28, GGCAAGTGGCCTACACTCATTTTGAAG (SEQ ID NO:29) and GAGTGTAGGCCACTTGCCAATGATCT (SEQ ID NO:30).

In embodiments, a nucleic acid sequence encoding one or more Mason-Pfizer monkey virus constitutive transport elements (CTE) is downstream of the nucleic acid sequence encoding the gag and gag-pol polyproteins in the second plasmid. This advantageously enables rev-independent expression of gag and gag-pol proteins.

In some embodiments a nucleic acid sequence encoding at least two CTEs is downstream of the nucleic acid sequence encoding the gag and gag-pol polyproteins. An exemplary nucleic acid sequence encoding two CTEs is SEQ ID NO:23. The inventors have surprisingly found that the inclusion of a nucleic acid sequence encoding two, three or four CTEs in the second plasmid provides increased efficiency of lentiviral vector particle production, relative to the inclusion of a nucleic acid sequence encoding one CTE.

In embodiments the second plasmid comprises a nucleic acid sequence encoding a CMV enhancer. A CMV enhancer is a known nucleic acid sequence which enhances expression of the nucleic acid for transfer from the vector system. An exemplary CMV enhancer nucleic acid sequence is provided by SEQ ID NO:31.

The second plasmid may comprise a nucleic acid sequence encoding a CAG promoter. The CAG promoter is formed from the following nucleic acid sequences:
(C) the cytomegalovirus (CMV) early enhancer element;
(A) the promoter, the first exon and the first intron of chicken beta-actin gene; and
(G) the splice acceptor of the rabbit beta-globin gene

An exemplary nucleic acid sequence encoding a CAG promoter is provided by SEQ ID NO:32. It will be appreciated that SEQ ID NO:32 comprises SEQ ID NO:31. In embodiments the CAG promoter is located upstream of the nucleic acid sequence encoding the gag polyprotein and the gag-pol polyprotein. This advantageously further improves the transduction efficiency of the lentiviral vector particles generated from the vector.

In embodiments the second plasmid comprises a nucleic acid sequence encoding a chimeric intron. SEQ ID NO:33 is an example of a nucleic acid sequence encoding a chimeric intron.

The second plasmid may further comprise a Kozak sequence. As the skilled person will appreciate, a Kozak sequence is a nucleic acid sequence which facilitates the initiation of translation of a target mRNA, for example the mRNA encoding the gag and gag-pol polyproteins. SEQ ID NO:34 is an exemplary Kozak sequence.

The second plasmid may comprise a pciNeo plasmid (SEQ ID NO:35) backbone. The pciNeo plasmid backbone is commercially available from Promega Corporation, Madison, Wis., USA. It will be appreciated that the backbone may be modified to include one or more of the nucleic acid sequences described herein.

An exemplary second plasmid sequence is SEQ ID NO:36. SEQ ID NO:36 may otherwise be referred to herein as a pCAG-VMV-GAgPol-CTE2X plasmid. SEQ ID NO:36 comprises a nucleic acid sequence encoding a CAG promoter upstream of the nucleic acid sequence encoding the gag polyprotein and the gag-pol polyprotein. SEQ ID NO:36 further comprises a nucleic acid sequence encoding two CTEs.

Further exemplary second plasmid sequences may be provided by SEQ ID NO:37 (which may otherwise be referred to herein as pCAG-VMV-GagPol-IN1) or SEQ ID NO:38 (which may otherwise be referred to herein as pCAG-VMV-GagPol-IN2). SEQ ID NO:37 comprises SEQ ID NO:36, wherein the nucleic acid sequence encoding the integrase comprises an E154A mutation. SEQ ID NO:38 comprises SEQ ID NO:36, wherein the nucleic acid sequence encoding the integrase comprises a D66A and a E154A mutation.

The nucleic acid encoding an envelope protein of the third plasmid may be derived from a vesicular stomatitis virus (VSG). Using an envelope protein derived from the VSG results in increased host tropism for the lentiviral vector particle(s) produced from the vector. An envelope protein derived from the VSG also improves the stability of the lentiviral vector particle(s) produced from the vector system and allows the lentiviral vector particles produced from the vector system to be easily isolated, for example by ultracentrifugation.

Other suitable envelope proteins are well known in the field. These include, but are not limited to, the SRLV Env protein, baculovirus gp64, and other viral glycoproteins. An exemplary third plasmid may be provided by the commercially available pMD2.G plasmid (Addgene, MA, US). Other commercially available plasmids suitable as the third plasmid will be known to the skilled person.

The fourth plasmid may further comprise a nucleic acid sequence encoding a promoter. The nucleic acid sequence encoding a promoter may be upstream of the nucleic acid sequence encoding the rev protein. In some embodiments the promoter comprises a CMV promoter.

In some embodiments the nucleic acid sequence encoding the Rev protein is a small ruminant lentivirus nucleic acid sequence, optionally a visna/maedi virus (VMV) nucleic acid sequence.

An exemplary fourth plasmid may be provided by the nucleic acid sequence of SEQ ID NO:39. SEQ ID NO:39 may otherwise be referred to herein as a pCMV-VMV-Rev plasmid. SEQ ID NO:39 comprises a CMV promoter and a visna/maedi virus nucleic acid sequence encoding a Rev protein. The fourth plasmid may comprise a pEGFP-C1 backbone, which is commercially available from Promega. The EGFP coding region of the backbone may be replaced with the nucleic acid sequence encoding a Rev protein.

It will be appreciated that any of the nucleic acid sequences described herein may be codon optimised or codon-modified. For example, the nucleic acid sequence encoding the Rev protein may be codon-optimised or codon-modified. Methods of codon optimisation and modification are available and known to those skilled in the art.

The vector system provided by this invention may comprise or further comprise a nucleic acid sequence encoding a reporter gene. Any of the first, second, third and/or fourth plasmids may comprise a nucleic acid sequence encoding a reporter gene. In embodiments, the first plasmid comprises a nucleic acid sequence encoding a reporter gene. The reporter sequence may encode a gene or peptide/protein, the expression of which can be detected by some means. Suitable reporter sequences may encode genes and/or proteins, the expression of which can be detected by, for example, optical, immunological or molecular means. Exemplary reporter sequences may encode, for example, fluorescent and/or luminescent proteins. Examples may include sequences encoding firefly luciferase (Luc: including codon-optimised forms), green fluorescent protein (GFP), red fluorescent protein (dsRed). An exemplary GFP nucleic acid sequence is provided by SEQ ID NO: 16.

Small ruminant lentiviruses are a small group of lentiviruses which are associated with clinical disease such as maedi, visna, arthritis and encephalitis in sheep and goats. The group comprises the visna/maedi virus (VMV) and caprine arthritis encephalitis virus (CAEV). Thus, in embodiments, the small ruminant lentivirus is selected from visna/maedi virus (VMV) and caprine arthritis encephalitis virus (CAEV). The small ruminant lentivirus may be visna/maedi virus (VMV).

It will be appreciated a nucleic acid sequence for transfer may be inserted into the insertion site of the first plasmid. Any suitable nucleic acid for transfer may be envisaged by the skilled person. For example, the nucleic acid for transfer may comprise a gene encoding an immunogen, for example one or more pathogen genes relevant to a disease of interest. This may comprise, for example, a viral surface glycoprotein. In some embodiments, the nucleic acid for transfer may encode a wild-type version of a gene known to be mutated in a subject, for example a gene encoding a cystic fibrosis transmembrane receptor. In other embodiments, the nucleic acid for transfer may comprise a gene which the user wishes to overexpress in a host cell, for example for purification and experimental use by the user. Thus, the nucleic acid for transfer may comprise a gene encoding a hormone or another protein which the user wishes to investigate experimentally. It will also be appreciate that the nucleic acid for transfer may encode genes for use in CRISPR, guide RNAs and/or Cas9 nuclease.

A composition or vaccine of this invention may be formulated as a sterile composition and may comprise one or more excipients, carrier and/or diluents—for example one or more pharmaceutically acceptable excipients, carrier and/or diluents.

In embodiments the immunogenic composition or vaccine further comprises or is admixed with an antigen, a polypeptide and/or an adjuvant.

The compositions and vaccines of this invention may be formulated for oral, topical (including dermal and sublingual), intramammary, parenteral (including subcutaneous, intradermal, intramuscular and intravenous), transdermal and/or mucosal administration. In embodiments the compositions and vaccines of this invention may be formulated for parenteral administration, optionally subcutaneous, intradermal, intramuscular and/or intravenous administration.

### DETAILED DESCRIPTION

The present invention will now be described in detail with reference to the following Figures which show:

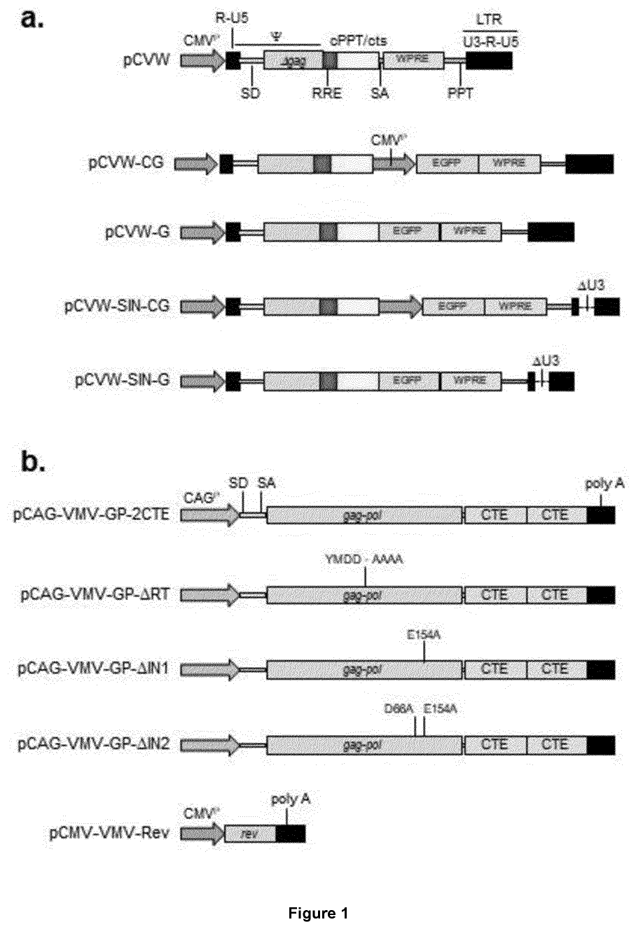

FIG. 1. Plasmids used for VMV vector production: The figure illustrates the features of the various expression plasmids used in this study. (a) VMV transfer vector constructs. CMV^{P}, human cytomegalovirus immediate early promoter; R, repeat region of VMV long terminal repeat (LTR); U5, unique 5 region of VMV genome; U3, unique 3 region of VMV LTR; SD, Splice donor; SA, splice acceptor; putative encapsidation element; Agag, partially deleted region of VMV gag gene; RRE, VMV Rev responsive element; cPPT/cts, proposed VMV central polypurine tract and central termination sequence (two of these were tested as described in main text); EGFP, enhanced green fluorescent protein; WPRE, woodchuck hepatitis virus post-transcriptional regulatory element; U3, deletion of 147 bp (SIN1) or 173 bp (SIN2) of the VMV U3 region. (b) Packaging plasmids. CAGE, chicken beta-actin/CMV promoter/enhancer element; CTE, MPMV constitutive transport element; poly-A, SV40 late polyadenylation signal. Sites of mutations introduced to create reverse transcription (ΔRT) and integration (ΔIN)-defective vectors are shown.

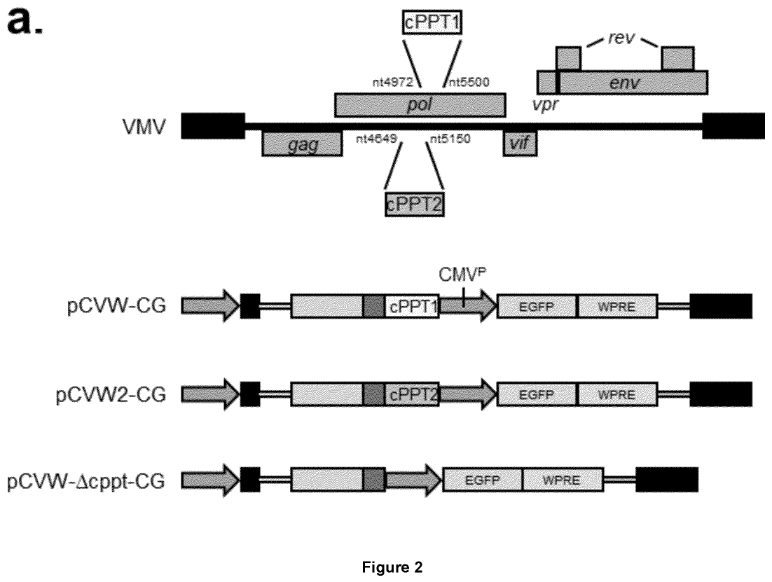

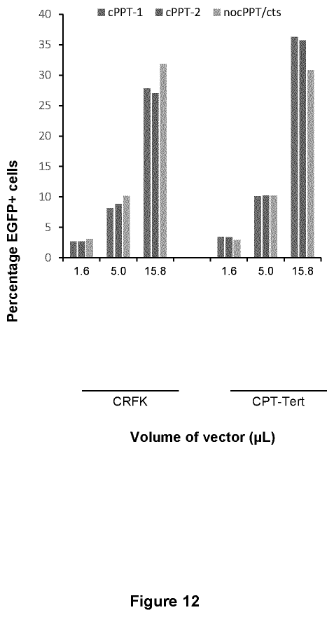

FIG. 2: Sequences of the putative VMV cPPT/cts elements: The position of the two putative cPPT/cts elements relative to the VMV genome (strain KV1772) is shown. All experiments used cPPT1 except for those shown in FIG. 12.

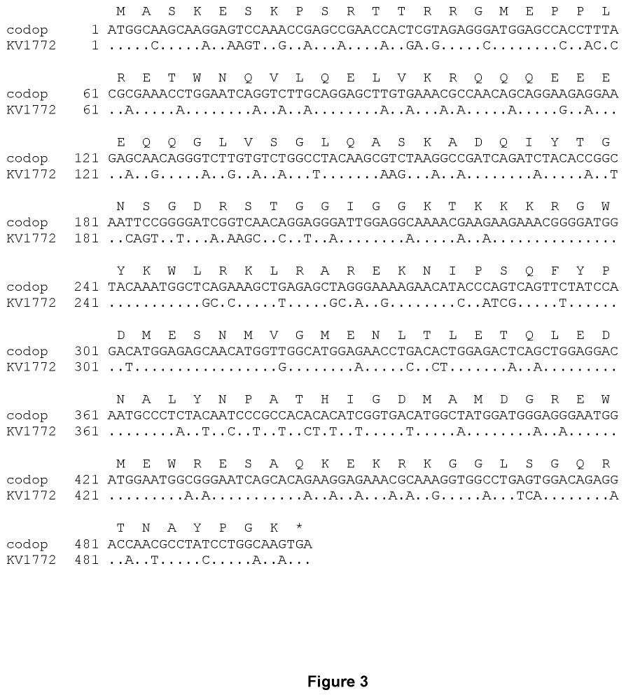

FIG. 3: Sequence of codon optimized VMV Rev: The nucleotide sequence of the human codon-optimized VMV Rev used in this study (codop) is shown aligned with the sequence of the Icelandic KV1772 strain (Genbank accession S55323). Dots (.) indicate identity with the codon-optimized sequence and nucleotide differences are shown. The amino acid sequence is shown above the nucleotide alignment.

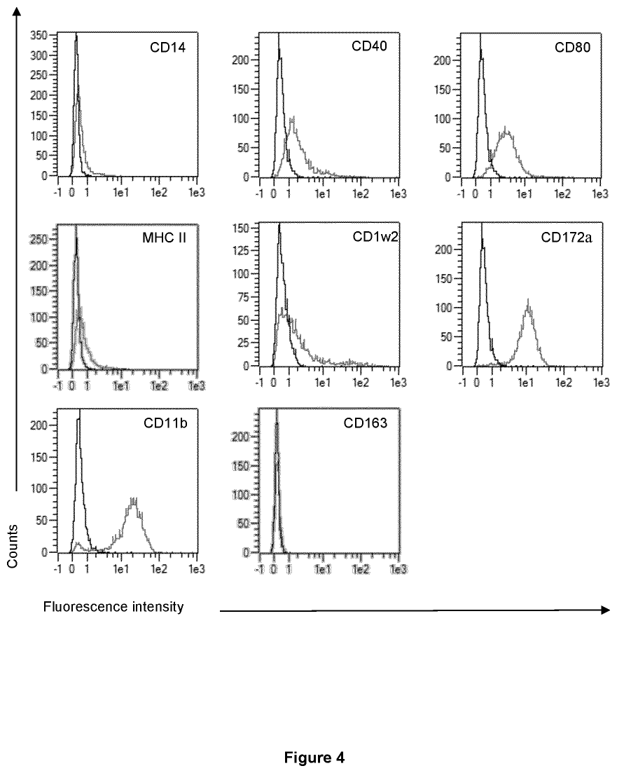

FIG. 4: Phenotyping of ovine MDDCs: Expression of cell surface molecules on ovine MDDCs. The black histograms represent the isotype-matched controls and the red histograms represent cell surface molecules (CD14, CD40, CD80, MHC II, CD1w2, CD172a, CD11b and CD163). The histograms are from one sheep and are representative of the profiles observed in three sheep. Ten thousand cells were counted.

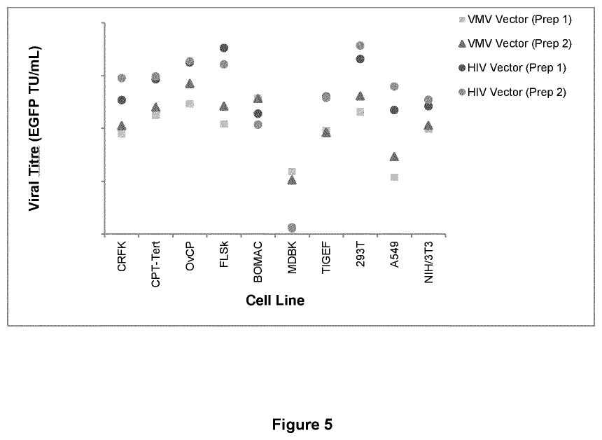

FIG. 5: Transduction of cell lines with lentiviral vectors derived from VMV and HIV-1: VMV and HIV-1 lentiviral vectors were produced in 293T cells by transient transfection and applied to the indicated cell lines. EGFP-positive cells were measured by flow cytometry 72 hours post-transduction (50,000 cells were measured) and vector titres were calculated from the average of three replicate transductions on each cell line. Titres (EGFP-transducing units/mL) are shown for two independent preparations of each vector. The detection limit of the assay was 1×10³ EGFP-transducing units/mL

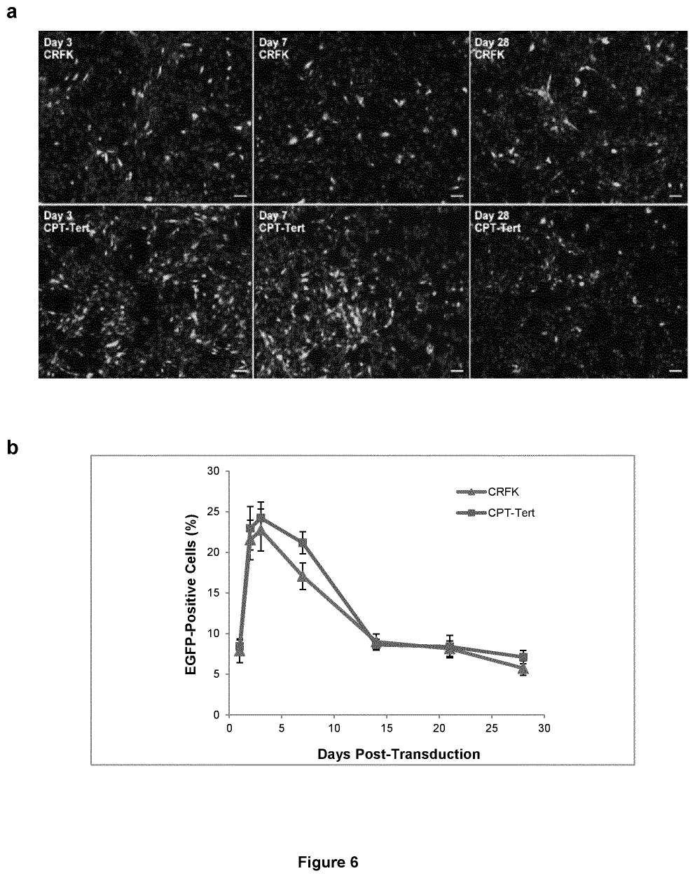

FIG. 6: Time course of transduction of cell lines by VMV lentiviral vectors: CRFK and CPT-Tert cells were transduced with VMV vectors carrying a CMV-EGFP expression cassette (CVW-CG) at an MOI of 0.25 and analyzed by flow cytometry for the percentage of EGFP-positive cells at intervals up to 28 days post-transduction. (a) Fluorescent images of CRFK and CPT-Tert cells at 3, 7 and 28 days post-transduction. Green indicates EGFP, blue indicates DAPI staining of nuclei. Scale bars represent 50 μm. (b) Fifty thousand cells were measured for EGFP fluorescence by flow cytometry at each time point. The data shown represent the mean of experiments with three independent vector preparations each performed in triplicate. Error bars indicate standard deviation.

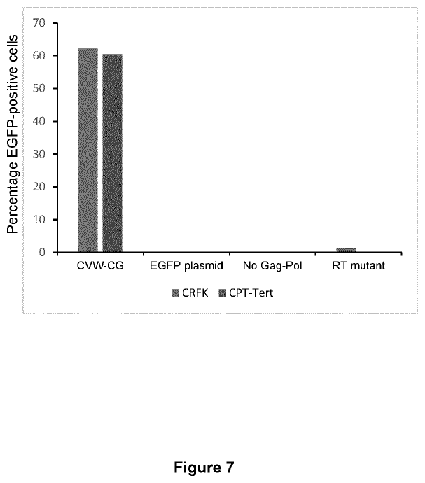

FIG. 7: EGFP-positive cells are a result of vector-mediated transduction and not direct transfer of EGFP from producer cells: CRFK and CPT-Tert cells were transduced with VMV lentiviral vectors (CVW-CG) or with unconcentrated supernatants from control transfections in which, (i) pEGFP-C1 replaced pCVW-CG (‘EGFP plasmid’); (ii) the packaging plasmid was omitted and replace with an empty expression plasmid (‘No Gag-Pol’); (iii) vectors prepared with a defective packaging plasmid (‘RT mutant’). Three days post-transduction, the percentage of EGFP-positive cells was measured by flow cytometry. Fifty thousand cells were measured from each sample.

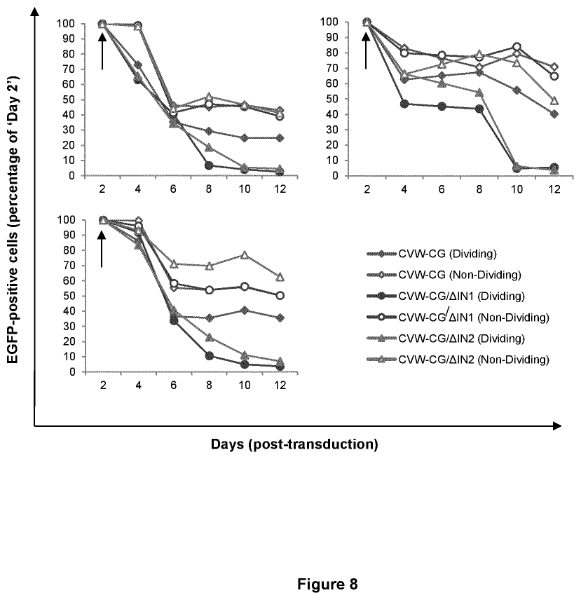

FIG. 8: Transduction of arrested CRFK cells with integration-defective VMV vectors: CRFK cells were arrested using 15 μg/mL aphidicolin 24 hours pre-transduction (day −1). On day 0, cells were transduced with integration-competent CVW-CG and two integration-defective vectors: CVW-CG/ΔIN1 and CVW-CG/ΔIN2 at an MOI of 1. After 48 hours, one duplicate well of arrested cells was released from cell cycle arrest and allowed to resume cycling for the remainder of the experiment (marked by black arrows). The percentage of EGFP-positive cells was then determined by flow cytometry every two days until day 12. Fifty thousand cells were measured at each time point from three independent experiments. Each graph shows data from an individual experiment and shows the mean of two technical repeats as a percentage of the Day 2 values. The percentage of EGFP-positive cells for these experiments are shown in Table 2. Closed symbols indicate dividing cells, open symbols indicate arrested cells.

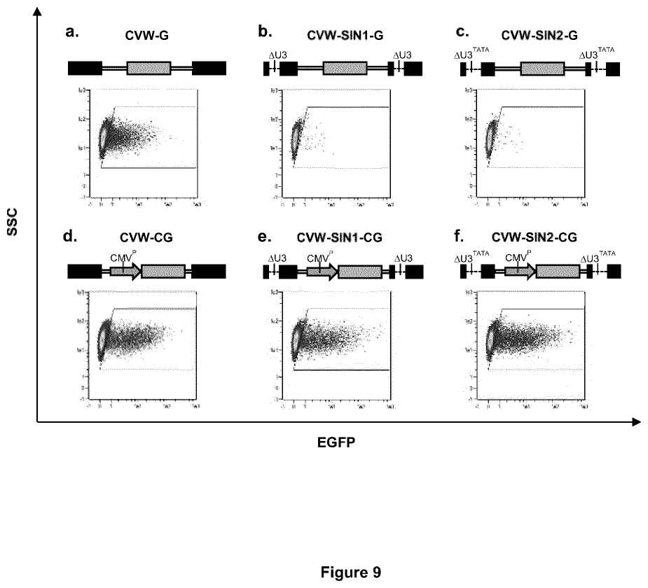

FIG. 9: Transduction of ovine choroid plexus cells with self-inactivating VMV vectors: Ovine choroid plexus (SCP) cells were transduced with the indicated VMV lentiviral vectors encoding EGFP. Input volumes were standardized to CVW-CG (MOI of 0.2 on CRFK cells) based on the amount of mature CA protein determined by immunoblotting. Cells were analyzed by flow cytometry for EGFP fluorescence 72 hours post-transduction. Fifty thousand cells were measured from each sample. The experiment was repeated at least three times and the figure shows the results of one representative experiment. The expected structures of the reverse-transcribed vector products are shown above each plot. (a) CVW-G: transgene expression is driven by the VMV LTR. (b) CVW-SIN1-G: a deletion was created in the U3 region of the LTR to remove transcriptional control elements and enhancers in the viral LTR; transgene expression was reduced. (c) CVW-SIN2-G: a larger deletion was created in the U3 region of the LTR that encompassed the TATA box; transgene expression was reduced. (d) CVW-CG: transgene expression is driven by the internal CMV promoter. (e) CVW-SIN1-CG: the internal CMV promoter allows transgene expression even where the LTRs are non-functional. (f) CVW-SIN2-CG: the internal CMV promoter again restores transgene expression, in this instance exceeding the efficiency of the parental vector.

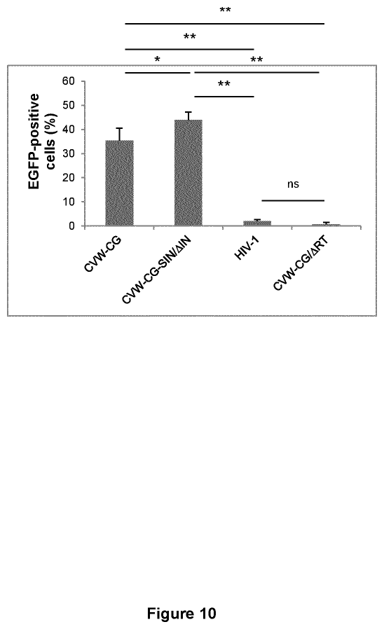

FIG. 10: VMV lentiviral vectors transduce primary ovine MDDCs more efficiently than HIV-1 vectors: Ovine MDDCs were transduced with VMV and HIV-1 lentiviral vectors at an MOI of 1 (determined on CRFK cells). The amount of CVW-CG/ART used was standardized against CVW-CG using immunoblotting for VMV CA. EGFP-positive cells were analyzed 72 hours post-transduction by flow cytometry (50,000 cells were counted). Differences in the percentage of EGFP-positive cells between vector constructs were calculated using the Mann-Whitney Test (* p≤0.05; ** p≤0.01; ns: no significance). Error bars represent standard deviation (n=4 sheep).

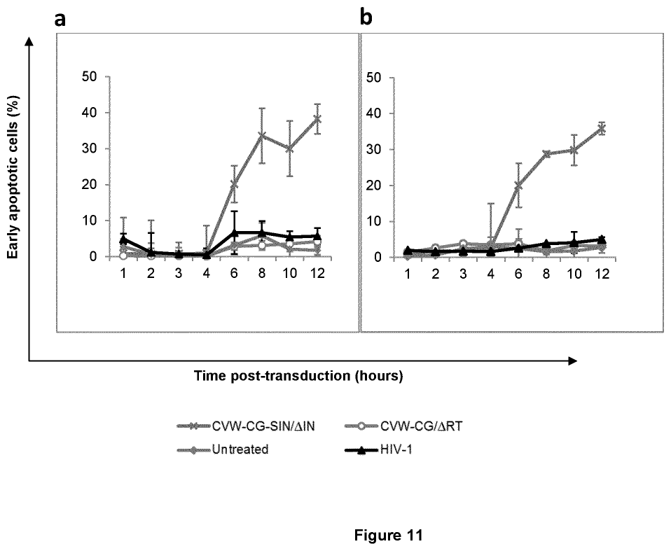

FIG. 11: VMV vectors induce apoptosis in ovine MDDCs: Cells were transduced at an MOI of 1 (determined on CRFK cells). The amount of CVW-CG/ART added was standardised against CVW-SIN2-CG/ΔIN by immunoblot assay for VMV CA. Cells were harvested at various time points until 12 hours post-transduction. Ten thousand events were counted at each time point and analyzed using MACSQuantify software. Panels (a) and (b) show data from ovine MDDCs from two different sheep. Early apoptotic cells were defined as those positive for Annexin V staining but negative for 7-AAD. Error bars represent the standard deviation of three technical repeats.

FIG. 12: Inclusion of the VMV cPPT/cts does not increase VMV vector titre: CRFK and CPT-Tert cells were transduced with the indicated volume of VMV lentiviral vectors containing cPPT-1, cPPT-2 or no cPPT. The vector stocks were 25× concentrated and standardized by RT activity prior to plating on to cells. Three days post-transduction, the percentage of EGFP-positive cells was measured by flow cytometry. Fifty thousand cells were measured from each sample. The experiment was repeated at least three times, the results of one representative experiment are shown.

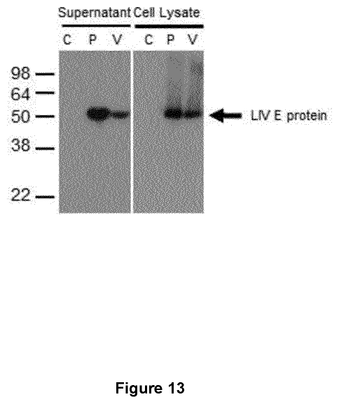

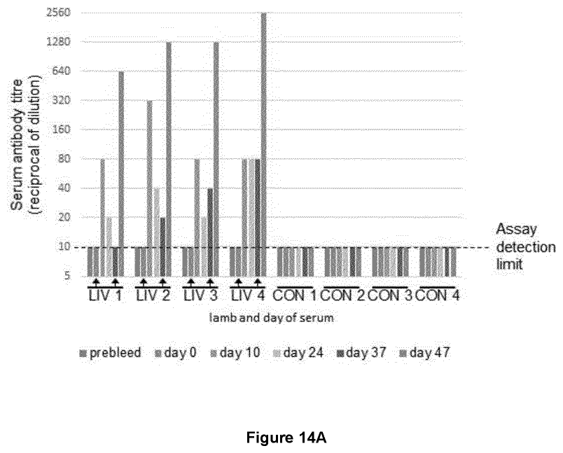

FIG. 13: Expression of LIV prME detected by immunoblot: 293T cells were transfected with CVW-LIV-prME plasmid (P), transduced with CVW-LIV-prME lentiviral vectors (V) or left untreated (control, C). Protein extracts were prepared 2 days (P) or 4 days (C and V) later from culture supernatants (left panel) and from cell lysates (right panel) and prME protein detected by immunoblotting using a pool of 2 monoclonal antibodies to the LIV E protein. A band of approximately 50 kDa in transfected and transduced cells indicates successful expression of the protein.

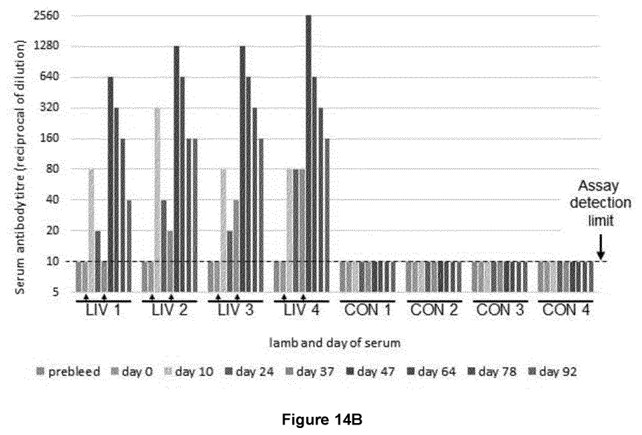

FIGS. 14A & B: Antibody response LIV in sheep receiving CVW-LIV-prME ovine lentiviral vectors (LIV 1-4) and in unvaccinated control sheep (CON 1-4): Antibodies were measured by hemagglutination inhibition. The y-axis shows the reciprocal of the highest dilution of each serum that neutralised. The x-axis indicates the individual sheep and the day each serum sample was tested. Vertical arrows indicate the days on which lentiviral vector was administered. The prebleed was taken 18 days prior to the day of the first vaccination (day 0). The detection limit of the assay is indicated by the horizontal dotted line. The data show that the four lambs that received the CVW-LIV-prME vector produced antibodies to LIV, whereas the four sheep that received no vector did not produce antibodies to LIV.

### EXAMPLE 1

### Summary

In this study, we developed a gene transfer system from VMV that is capable of efficient transduction of cultured cell lines from a range of species, including sheep, cattle and humans. In addition, integration-defective and self-inactivating vectors were produced with only a modest reduction in infectious titre. Notably, the VMV vectors infect ovine monocyte-derived dendritic cells (MDDCs) more efficiently than vectors derived from HIV-1, although we also found that VMV vectors rapidly induce apoptosis in these cells. This study demonstrates that efficient gene transfer vectors can be produced from SRLV.

### Materials and Methods

### Construction of Vector Plasmids

The pCVW vector (SEQ ID NO:18)(FIG. 1a) was designed in silico, prepared by gene synthesis (MWG Eurofins) and subcloned into the Not I and Sal I sites of pBluescript. The EGFP coding sequence was isolated from pEGFP-C1 (Clontech) by PCR (primers: CCGGTCGCCACCATGCATAGCAAGG and GACTGCAGAATTCGAAGCTTGAGC), digested with Nsi I and Sfu I and subcloned into Nsi I/Sfu I-digested pCVW to create pCVW-G. An Nsi I-Sfu I fragment encoding a CMV-EGFP expression cassette was excised from pEGFP-C1 and inserted into Nsi I/Sfu I-digested pCVW to create pCVW-CG (SEQ ID NO:19).

Self-inactivating VMV vector plasmids were produced by removing a region of the 3′ U3 region containing enhancer sequences and promoters from pCVW-CG. pCVW-SIN1-CG (SEQ ID NO:21) was created by linearising by PCR (primers: TTAAGTGACATGACCTTCCTATAACTC (SEQ ID NO:40) and TTAAGTAAACAAGTTGCCTATATAAGC (SEQ ID NO:41)) and re-ligating to recircularize. (This also introduced a PacI site into the plasmid.) pCVW-SIN2-CG (SEQ ID NO:22) was created by cloning a 200 bp synthetic gene (MWG-Eurofins) into the Sal I and Pac I restriction sites in pCVW-SIN1-CG (SEQ ID NO:21).

To delete the putative cPPT/cts from the VMV vector, pCVW-CG (containing cPPT-1, SEQ ID NO:42) was digested with HpaI and BamHI, blunt-ended with KOD high fidelity DNA polymerase (Merck) and religated to create pCVW-cPPT-CG. To generate pCVW2-CG, which contains an alternative putative cPPT/cts (FIG. 2, SEQ ID NO:43), a synthetic gene fragment encoding this region was substituted into the HpaI and BamHI sites of pCVW-CG.

The VMV Gag-Pol expression plasmid (FIG. 1b) was created by PCR amplification from DNA from the blood of an infected sheep (strain EV1⁵⁴) with primers GATCGATCGTCGACAGTGCCACCATGGCGAAGCAAGGCTCAARRGAG (SEQ ID NO:44 and GATCGATCGCGGCCGCGGCAACCGAGGCCCTATCTCCCTA (SEQ ID NO:45). The PCR products were digested with Sal I and Not I and inserted into pCAGneo (a derivative of pCIneo (Promega) that contains a chicken beta-actin/CMV enhancer/promoter element). Two copies of the MPMV CTE²⁷ were then inserted downstream of the coding region to generate plasmid pCAG-VMV-GP-2CTE (SEQ ID NO:36). The sequence of the clone used in the experiments described here is SEQ ID NO:24. SEQ ID NO:24 encodes the gag polyprotein amino acid sequence (SEQ ID NO:46) and the gag-pol polyprotein amino acid sequence (SEQ ID NO:47). Mutations were subsequently introduced into the IN domain of pCAG-VMV-GP-2CTE using Gibson assembly. For the E154A mutation, primers were INdel-F1 (GGCAAGTGGATTACACTCATTTTGAAG, SEQ ID NO:25), INdel-R (CCTGGCCACTAGAGCTTGAGACTGTGG, SEQ ID NO:26), INdel-Plas-R1 (GAGTGTAATCCACTTGCCAATGATCT, SEQ ID NO:27) and INdel-Plas-F (CAAGCTCTAGTGGCCAGGGCTCATCAG, SEQ ID NO:28). For the D66A/E154A dual mutation, primers were INdel-F2 (GGCAAGTGGCCTACACTCATTTTGAAG, SEQ ID NO:29), INdel-R (SEQ ID NO:26), INdel-Plas-R2 (GAGTGTAGGCCACTTGCCAATGATCT, SEQ ID NO:30) and INdel-Plas-F (SEQ ID NO:28). The PCR fragments were ligated using Gibson Assembly Cloning (NEB) following the manufacturer's guidelines. A ΔRT mutant Gag-Pol plasmid was generated by PCR of pCAG-VMV-GP-2CTE (primers: GGGATAGCTGCTGCCGCTATCTATATAGGC (SEQ ID NO:48) and CTATATAGATAGCGGCAGCAGCTATCCCAAATTG (SEQ ID NO:49)) to create a template for a second PCR (primers: CAAGGACATCTTGCAAGACAATGCAGG (SEQ ID NO:50) and CGGTGGAAGCAATATATCCTAAGCTTCCTTC (SEQ ID NO:51)). The PCR fragment and pCAG-VMV-GP-2CTE were digested with Pml I and ligated to create pCAG-VMV-GP-RT.

To generate a VMV Rev expression plasmid (SEQ ID NO:39, pCMV-VMV-Rev), a synthetic gene fragment encoding human codon-optimized VMV Rev was subcloned into the Nhe I and BamHI sites of pEGFP-C1. This removes the EGFP coding sequence and places Rev downstream of the CMV promoter element. The sequence of the codon-optimized VMV Rev used here is SEQ ID NO: 52. The amino acid sequence encoded by SEQ ID NO:52 is SEQ ID NO:53. SEQ ID NO:52 is also shown in FIG. 3 aligned with the sequence of the Icelandic KV1772 strain (Genbank accession S55323). All PCR reactions were performed using KOD high fidelity DNA polymerase (Merck) and the sequences of all plasmids were verified by DNA sequencing (MWG Eurof ins Genomics).

HIV-1 SIN lentiviral vectors were prepared using pCS-CG³³ (Addgene plasmid #12154; kindly provided by Inder Verma), pMDLg/pRRE, pMD2.G² (Addgene plasmids #12251 and #12259; both kindly provided by Didier Trono) and pCNC-Rev⁷² (a kind gift from Yasuhiro Takeuchi).

### Cell Culture

293T and CPT-Tert⁷³ cells were cultured in Iscove's modified Dulbecco's medium supplemented with 10% fetal calf serum (FCS) (Sigma) and 4 mM glutamine. CRFK⁷⁴, A549⁷⁵, MDBK⁷⁶, TIGEF⁷⁷ and NIH/3T3 cells were grown in Dulbecco's modified Eagle's medium supplemented with 10% FCS, 2 mM glutamine and 1% non-essential amino acids (NEAA) (Sigma). Primary sheep choroid plexus (SCP) and fetal lamb skin (FLSk) cells (both Moredun Research Institute) were cultured in 199 medium (Sigma) supplemented with 10% FCS, 2 mM glutamine, 10% tryptose phosphate broth and 2% sodium bicarbonate. BOMAC cells⁷⁸ were cultured in RPMI 1640 medium supplemented with 2 mM glutamine and 10% FCS.

### Lentiviral Vector Production and Transduction

Lentiviral vector particles were prepared by 4-plasmid transfection of 293T cells in T75 flasks using FuGENE HD (Promega) as recommended. Briefly, cells at 80% confluence were transfected with 5.4 μg vector plasmid, 3.6 μg packaging (Gag-Pol) plasmid, 1.8 μg pMD2.G plasmid (which encodes VSV-G) and 1.2 μg Rev plasmid per flask. Medium was removed 18 hours later and replaced with 10 mL fresh medium supplemented with 5 mM sodium butyrate. Supernatant containing vectors was harvested 24 hours later and the cells re-fed (without sodium butyrate) and a further harvest made 24 hours after that. The two harvests were pooled and filtered (0.45 μm cellulose acetate; Sartorius) before storing at −80° C. Some vector preparations were concentrated by ultracentrifugation (35,000×g, 2 hours, 4° C.) and resuspended in serum-free medium before use (10×-50× concentration).

For the transduction of cells in 12-well plates, 1×10⁵ cells were plated in each well on the day prior to transduction. Immediately before transduction, medium was removed from the cells and replaced with 500 μL of medium containing lentiviral vectors with 8 μg/mL polybrene (Sigma-Aldrich). The number of cells per well was counted at the time of vector addition and used to calculate vector titer. Medium containing viral vectors was removed 4 hours later and replaced with fresh medium. Ovine MDDC were transduced as for cell lines except that cells were plated in 96-well plates (1×10⁵ cells/well) and transduced in a total volume of 200 μL.

### RT Activity Assays

Reverse transcriptase activity was assessed using a colorimetric reverse transcriptase assay (Roche) according to the manufacturer's protocol and the assay read on an iEMS Reader MF (Labsystems). The concentration of reverse transcriptase activity in each vector preparation was calculated from standard curves generated from known amounts of HIV-1 RT and expressed as ng/mL. These values were used to normalize vector preparations prior to in vitro transduction. For CVW-CG/RT, we standardized against RT-competent vector stocks by immunoblot using a rabbit anti-VMV CA polyclonal antibody and used an appropriate volume for transductions.

### Flow Cytometry

Cell lines were detached from culture plates by treatment with 0.0125% trypsin/3.2 mM EDTA, or with TrypLE Express (Life Technologies; MDDC only) before washing in PBS (400 g for 10 minutes at 20° C.) and re-suspending at approximately 1×10⁶ cells/mL in PBS. LIVE/DEAD Fixable Violet stain (Life Technologies) (14 per 10⁶ cells) was added to the cell suspension and incubated at room temperature for 30 minutes protected from light. Cells were washed once with PBS before final suspension in 1% paraformaldehyde. EGFP-positive cells were acquired on a MACSQuant Analyser and the results analysed using MACSQuantify software. To calculate infectious titres, the percentages of EGFP-positive cells were used in the following equation: IFGFcFiGGETiFrF (TE/EL)=((AHFraHF EGFP %−HI)×DF)×(N^{o} iFGFcFFE cFllE); where HI is the value obtained with the heat inactivated vector and DF in the dilution factor of the vector required to calculate titre per 1 mL

For the majority of experiments, the percentage of EGFP-positive cells was below 20% and therefore within the range where the relationship between MOI and infectious titre is close to linear. For a small number of vector stocks (e.g., HIV-1 from FIG. 5), greater than 20% EGFP-positive cells were observed. In these cases this linear relationship does not apply so to avoid underrepresenting the ‘true’ infectivity a correction was made to estimate the ‘true’ MOI from the percentage of EGFP-negative cells using the formula m=−ln P(0) derived from the Poisson distribution, as employed by Grigorov and colleagues⁷⁹ (where P(0) is the fraction of cells that are EGFP-negative cells and m is the ‘true’ MOI).

### Generation of Ovine MDDCs

Sheep PBMCs were obtained from whole blood collected in citrate phosphate dextrose adenosine-1 blood bags (Henry Schein). Buffy coats were underlaid with Lymphoprep (Axis Shield) and interface cells collected after density centrifugation at 1200×g for 25 minutes. PBMCs were then washed three times with phosphate-buffered saline (PBS) to remove platelets. CD14⁺ cells were positively selected using magnetic separation (Miltenyi Biotech) according to the manufacturer's guidelines and cultured in IMDM supplemented with 10% FCS at a density of 1×10⁶ cells/mL in ovine ‘DC mix’. DC mix is an optimised, in-house formulation comprising recombinant interleukin (IL)-4 and granulocyte-macrophage colony stimulating factor (GM-CSF) expressed in Chinese Hamster Ovary (CHO) cells for differentiation of monocytes to DC based on the phenotypic characterisation described below. After 3 days, 1 mL of medium was removed and 2 mL fresh medium was added. At day 6, immature MDDCs were dissociated from the plastic using cell dissociation fluid (Sigma-Aldrich) for 1 hour at 37° C. Cells were then washed in medium, and re-plated in the relevant plate format for transduction and left to re-adhere overnight. The phenotype of these cells was confirmed by flow cytometry using a panel of monoclonal antibodies specific for cell surface molecules (FIG. 4; Table 1). All procedures using sheep were performed with approval from the Animal Welfare and Ethical Review Body of the Moredun Research Institute in accordance with the U.K. Animals (Scientific Procedures) Act 1986.

### Phenotyping of Ovine MDDC

Cells were suspended in PBS before the addition of LIVE/DEAD Fixable Violet stain (Life Technologies) (1 μL per 1×10⁶ cells) and incubated for 30 minutes protected from light. The cells were then washed with PBS and centrifuged at 300×g for 10 minutes. Cells were resuspended in 20% normal goat serum (Merck Millipore) in PBS and blocked for 30 minutes in round-bottomed 96-well plates. The plates were washed once with 100 μL FACs buffer (5% FBS, 0.05% sodium azide in PBS) then twice with 200 μL PBS, centrifuging at 900×g for 30 seconds after each wash step. Primary antibody (50 μL) or the equivalent isotype control mAb was then added to the appropriate wells. Following incubation for 30 minutes at 4° C., the cells were centrifuged and washed as before. The secondary antibody was then added in 50 μL volumes and the plates incubated for a further 30 minutes. The plates were then washed in FACs buffer and finally with PBS. The cells were then fixed in 1% paraformaldehyde (Sigma-Aldrich) in PBS and analyzed using the MACSQuant Analyser (Miltenyi Biotec). Cell surface molecule markers used to assess the phenotype of ovine MDDCs were CD14, MHC class II, CD172a, CD40, CD80, CD1w2, CD11b and CD163 (Table 1).

### Measurement of Apoptosis and Necrosis in Ovine MDDCs

Cells were stained to differentiate cell death between apoptosis and necrosis using the Apoptosis/Necrosis detection kit from Enzo Life Sciences following the manufacturer's guidelines. Cells were suspended in 1× Binding Buffer, 1% Annexin V EnzoGold and 1% Necrosis Detection Reagent and incubated at room temperature for 15 minutes in the dark. Cells were then washed with PBS before final suspension in 1% paraformaldehyde and analysed by flow cytometry.

### Statistical Analysis

Statistics were performed using Minitab v.17 statistical software. Where data were normally distributed or could be transformed to be, 2-sample T-tests were utilized to determine significant differences between two groups where one outcome was measured. Where data were not normally distributed and could not be normally transformed, a Mann-Whitney test was used to determine any differences between the median and the spread from that between two different groups.

### Results

Construction of a Lentiviral Vector System from Visna/Maedi Virus

A VMV transfer vector plasmid (designated pCVW, SEQ ID NO:18) was designed that incorporates several features common to current ‘third generation’ lentiviral vectors^{1,2,22} (FIG. 1a). These include: (i) a hybrid human cytomegalovirus (CMV) immediate early promoter fused to the R and U5 regions of the VMV long terminal repeat (LTR) to enable high-level expression in 293T cells; (ii) a 1.2 kb region of the VMV 5 leader and gag region to provide a packaging signal for encapsidation into vector particles²³; (iii) the VMV Rev-responsive element²⁴; (iv) the woodchuck hepatitis virus posttranscriptional regulatory element (WPRE)²⁵; and (v) the VMV polypurine tract (PPT) and 3′ long terminal repeat (LTR). The major splice donor and acceptor sites of VMV were also retained and unique restriction sites were included downstream of the splice acceptor site for insertion of transgene expression cassettes. Some versions of the vector plasmid also included a putative VMV central polypurine tract/central termination sequence (cPPT/cts)²⁶. A CMV-EGFP expression cassette was inserted to generate pCVW-CG (FIG. 1a; SEQ ID NO:19) to allow assessment of the gene transfer activity function of the vectors.

A VMV packaging plasmid (pCAG-VMV-GP-2CTE, SEQ ID NO:36) was produced by inserting the VMV Gag-Pol coding region into a mammalian expression plasmid upstream of two copies of the Mason-Pfizer monkey virus (MPMV) constitutive transport element (CTE), which allows Rev-independent expression of Gag-Pol proteins²⁷ (FIG. 1b). A VMV Rev expression plasmid was prepared using a codon-optimized Rev sequence obtained by synthetic gene synthesis (FIG. 1b; SEQ ID NO:52). Co-transfection with this plasmid was predicted to enhance the production of infectious vector particles in transfected cells. For all experiments, vectors were pseudotyped with the vesicular stomatitis virus G protein (VSV-G)²⁸.

Ovine Lentiviral Vectors Transduce Cell Lines from a Variety of Species.

In initial experiments, VMV vector particles (denoted CVW-CG) were produced by transient transfection of 293T cells with the four vector plasmids (pCAG-VMV-GP-2CTE (SEQ ID NO:36), pCMV-VMV-Rev (SEQ ID NO:39), pCVW-CG (SEQ ID NO:19) and pMD2.G (encoding VSV-G)) and tested for their ability to transduce a range of human, ruminant, and rodent cell lines. Filtered supernatants were applied to cells and the percentage of EGFP-positive cells was measured 72 hours later by flow cytometry and used to calculate vector titre. The results indicate that this VMV vector has a broad tropism in vitro (FIG. 5) with most of the cell lines tested showing titres in the range 10⁵-10⁶ transduction units (TU) per mL. Exceptions were the bovine cell line MDBK and the human cell line A549, which showed lower titres (around 1×10⁴ TU per mL). For almost all the cell lines tested, infection with HIV-1 vectors was more efficient than VMV vectors (up to 10-fold). Exceptions were MDBK cells, where the percentage of HIV-transduced cells was close to the background of the assay, and the bovine macrophage cell line BOMAC, in which VMV vectors and HIV vectors had similar titres. Collectively, these experiments demonstrated that this VMV lentiviral vector system can produce infectious titres that approach those of HIV-1 vector systems.

A common potential artefact in assessing lentiviral gene transfer is the detection of ‘false positive’ cells that are not stably transduced. This has been attributed to the direct transfer of the transgene-encoded protein from the producer cell to the target cell) (pseudotransduction^{29,30} or to expression from episomal forms of the vector that have not integrated³¹. To study the stability of transduction by VMV vectors, CRFK and CPT-Tert cells were transduced with CVW-CG and expression of EGFP was measured at various time points up to four weeks post-transduction. Transduced cells were clearly visible in both cell lines (FIG. 6a). We found that the percentage of positive cells peaked at 72 hours post-transduction and then declined over time until two weeks after transduction when approximately 30-40% of the cells initially transduced remained EGFP-positive (FIG. 6b). After this, the proportion of positive cells remained stable for at least two weeks in both cell lines. Therefore, not all of the initial fluorescence observed following transduction is stably expressed.

To test for pseudotransduction, we prepared vectors according to the standard protocol but omitted the Gag-Pol packaging plasmid or substituted the transfer plasmid with a non-viral EGFP expression plasmid. Transfection of 293T cells with these plasmids should produce a high percentage of EGFP-positive cells but should not result in release of vector particles. We also prepared vectors using a Gag-Pol packaging plasmid that contained an inactivating mutation in pol (pCAG-VMV-GP-RT; FIG. 1b). Transfection of 293T cells using this packaging plasmid should result in the release of vector particles that are able to bind and enter target cells but are unable to reverse transcribe their RNA to DNA. All three control vectors resulted in low percentages (<1%) of EGFP-positive cells following plating on CRFK and CPT-Tert cells despite high level EGFP expression in the transfected producer cells (FIG. 7). This indicates that pseudotransduction by direct protein transfer does not account for the high level of initial EGFP-positivity observed, suggesting that the unstable fraction of the EGFP expression originates from unintegrated vector DNA.

### Generation of a Self-Inactivating, Integration-Defective Ovine Lentiviral Vector.

To improve the biosafety of the VMV vector system the system was modified using an approach that was guided by previous work on HIV-1 derived vectors^{13,32,33}. First, an integration-defective VMV vector was generated by introducing mutations into the packaging plasmid targeting the catalytic triad of the integrase coding region, which comprises three amino acids at positions D66, D118 and E154 (corresponding to positions D64, D116 and E152 of HIV-1 integrase)^{34,35}. These three residues are conserved across all retroviral and retrotransposon integrases and mutations at these sites in HIV-1 are known to impair integration with no apparent effect on other viral processes¹⁴.

To assess the infectivity of VMV integration-defective lentiviral vectors (IDLV), the pCAG-VMV-GP-2CTE (SEQ ID NO:36) packaging plasmid was modified to generate plasmids with single (E154A) and double (D66A/E154A) integrase mutations (ΔIN-1) and ΔIN-2 (SEQ ID NO:37 and SEQ ID NO:38, respectively; FIG. 1b) and used to prepare vector particles (denoted CVW-CG/ΔIN-1 and CVW-CG/ΔIN-2). VMV IDLVs were then plated onto duplicate plates of CRFK cells that had been arrested using aphidicolin 24 hours before transduction (day −1). On day 0, cells were transduced with VMV vectors at a multiplicity of infection (MOI) of 1. Two days post-transduction, the medium was replaced such that one replicate continued to be treated with aphidicolin whereas the other received medium without aphidicolin, thereby allowing the cells to re-enter the cell cycle. Subsequently, the cells were analysed by flow cytometry to measure the percentage of EGFP-positive cells every two days until day 12. At the end of this period, we found that in arrested cells, the percentage of EGFP-positive cells transduced with the integration-competent parent vector and the two forms of VMV IDLV were similar (approximately 50% of the day 2 values). In contrast, in the dividing CRFK cells a significantly lower percentage of EGFP-positive cells was seen for both IDLVs compared to the parental vector (p<0.001) (FIG. 8; Table 2). At day 12 post-infection, less than 4% of the IN1 and IN2 vector-transduced cells remained EGFP-positive, compared to over 30% of the cells transduced with integration-competent VMV vectors. Collectively, these results indicate that the two forms of VMV IDLV have infectivity similar to the integration-competent parental vector but the lower stability of expression in dividing cells suggests that the integration of these vectors was impaired. Notably, the vectors with the single and double mutated forms of integrase had similar activity in these experiments.

To generate self-inactivating (SIN) VMV vectors, the LTR region of the pCVW transfer plasmid was modified. Deletions introduced into the U3 region of the 3′ LTR are copied to the 5′ LTR during reverse transcription and therefore provide a means to eliminate the viral promoter in transduced cells^{36,37}. SIN vectors are predicted to reduce expression of lentiviral vector RNA in transduced cells and thereby minimize the generation of replication-competent lentivirus³³. Two SIN vectors were created: SIN1 (SEQ ID NO:21), which has a 147 bp deletion in U3 that removes binding sites for transcription factors important for activation of VMV transcription³⁸⁻⁴⁰; and SIN2 (SEQ ID NO:22), which also deletes the TATA box of the VMV promoter (173 bp deleted) (FIG. 1a). Vector plasmids were constructed with each SIN deletion, both with and without the internal CMV promoter driving EGFP expression (pCVW-SIN1/2-CG and pCVW-SIN1/2-G, respectively (FIG. 1a)) and used to prepare lentiviral vector particles. In order to test the infectivity of the SIN vectors, primary sheep choroid plexus cells (SCP, which are permissive for VMV and are known to support VMV LTR activity^{18,39} were transduced with SIN and non-SIN vectors and EGFP-positive cells were measured by flow cytometry 72 hours later. We found that both SIN deletions introduced into the VMV vector reduced EGFP expression in SCP cells to almost background levels (FIG. 9a-c), whereas inclusion of an internal CMV promoter restored EGFP expression to a level that was similar to or higher than that of the parental vector (FIG. 9d-f). These results indicate that the SIN VMV vectors can efficiently mediate gene transfer in SCP cells.

To determine the effect of combining SIN and IN mutations in the same vectors, vector stocks were prepared using the appropriate permutations of plasmids and their infectivity measured in CPT-Tert and CRFK cells. The vector stocks were analyzed for reverse transcriptase activity prior to transduction to allow standardization of the vector input. Flow cytometric analysis of transduced cells indicated that VMV vectors containing both SIN and IN mutations exhibited a decrease in transduction efficiency of between 3.88 and 4.81-fold compared to the parental vector CVW-CG (Table 3). Similar reductions in infectivity have been reported for SIN/IN mutants of other lentiviral vectors⁴¹⁻⁴⁴.

VMV Vectors Transduce Ovine Monocyte Derived Dendritic Cells More Efficiently than HIV-1 Derived Vectors

The experiments to this point showed that the VMV lentiviral vectors can efficiently transduce a variety of cell lines. We next tested the ability of CVW-CG and CVW-SIN2-CG/IN1 vectors to transduce primary ovine MDDCs. To monitor for pseudotransduction³⁰, we also transduced cells with vectors prepared using pCAG-VMV-GP-RT. For comparison, HIV-1 lentiviral vectors were prepared and tested in parallel. Vectors were first titrated on CRFK cells and then used to infect ovine MDDC at an MOI of 1. As before, transduction was measured by determining the percentage of EGFP-positive cells by flow cytometry 72 hours after plating vectors onto cells (FIG. 10). The two VMV vectors efficiently transduced ovine MDDC (between 30% and 45% of ovine cells EGFP-positive) and interestingly CVW-CG-SIN/ΔIN was found to transduce MDDCs more efficiently than CVW-CG (p≤3.05). Furthermore, the infectivity of the HIV-1 lentiviral vector on ovine MDDC was significantly lower than the two VMV vectors (2.0% cells infected) (p<0.01). The RT-defective control vector gave background levels of EGFP-positive cells. These results indicate that VMV lentiviral vectors are able to transduce primary ovine MDDC and do so more efficiently than vectors derived from HIV-1.

### The Ovine Lentiviral Vector Induces Apoptosis in Monocyte Derived Dendritic Cells

In the previous experiment testing susceptibility of ovine MDDC to VMV vectors, we noted that the morphology of transduced cells was altered in comparison to untreated cells indicating a cytopathic effect (data not shown). To investigate this further, ovine MDDCs were transduced with CVW-SIN2-CG/ΔIN1, CVW-CG/ART and a HIV-1-derived vector and subsequently harvested at specific time intervals up to 12 hours post-infection. The cells were stained with Annexin V and 7-aminoactinomycin D (7-AAD) and analysed by flow cytometry. Untransduced cells were also assayed at the same time-points. FIG. 11 shows the percentage of cells staining positive for Annexin V and negative for 7-ADD, which identifies cells in the early stages of apoptosis. The results show that greater than 30% of infected cells were in early apoptosis 12 hours post-infection compared to 5% of the untransduced cells. No increase in apoptosis was observed in cells transduced with the ΔRT VMV vector or the HIV-1 vector.

### Analysis of the Central Polypurine Tract of VMV Vectors

The experiments described above demonstrate that efficient gene transfer vectors can be derived from VMV in a similar way to that previously achieved for other lentiviruses, in contrast to previous work¹⁹. One difference between our vector system and that described previously is the inclusion here of a putative central polypurine tract/central termination element (cPTT/cts) within the vector genome. This cis-acting element produces a DNA ‘flap’ in the reverse-transcribed viral genome that has been shown in a number of studies to increase transduction of dividing and non-dividing cells by HIV-1 vectors most likely by increasing the efficiency of nuclear entry of the pre-integration complex.

For HIV-1, EIAV and FIV the sequences of the cPPT and cts have been determined⁴⁵⁻⁴⁵. In contrast, although a DNA flap has been mapped close to the centre of VMV genome⁴⁹, the precise cPPT/cts has not been defined experimentally. The genome of VMV has two polypurine motifs that potentially confer cPPT activity⁵⁰. In the experiments described above, the CVW vectors incorporated the downstream cPPT along with 500 bp of adjacent 5′ pol sequence that we predicted might contain the cts. To test whether this element is indeed responsible for the enhanced infectivity of the VMV vectors we compared the infectivity of this vector to one containing the upstream candidate cPPT (plus 500 bp 5′ flanking sequence; denoted CVW2-CG) and a vector with neither of these elements (FIG. 2). All three vectors showed similar infectious titres on CRFK and CPT-Tert cells (FIG. 12). Therefore, the inclusion of either putative cPPT/cts does not appear to confer a benefit to the infectivity of this vector system.

### Discussion

Lentiviral vectors are important tools for mediating gene transfer in vivo and in vitro. A number of systems have been developed from human, simian, feline, equine and bovine lentiviruses and these all transduce cells in vitro with high efficiency^{4-9,51}. In contrast, previous lentiviral vectors from SRLV have been shown to have low infectivity^{19-21.} In this study, we re-examined the ability of VMV to function as a lentiviral vector and the results demonstrate that it is possible to derive VMV vectors with infectious titres similar to those initially reported for other non-HIV vector systems⁵⁻⁹. In addition, we have shown that functional SIN and integration-defective VMV vectors can be constructed, with only a modest (up to 5-fold) reduction in infectivity. These vectors provide a new alternative to existing lentiviral vector systems that may offer advantages in some circumstances, in particular in studies in small ruminants.

Previous VMV vector systems¹⁹ gave very low transduction efficiencies on SCP and 293T cells. This was despite high levels of vector particle production and was attributed to cellular blocks to infection acting against VMV vectors during reverse transcription and/or integration¹⁹. It should be noted that the previous vectors were also assayed three days post-transduction so this difference is unrelated to the stability of EGFP expression. The vectors described in the present study have several differences in vector design that might contribute to the improved efficiency of gene transfer. These include the use of a 4-plasmid, ‘3^{rd} generation’ split genome design in which the VMV Rev protein is supplied in trans by a separate plasmid from that encoding Gag-pol; the use of the strong CAG enhancer-promoter in the packaging plasmid to drive Gag-Pol expression and the use of the WPRE.

We also investigated the effect of the inclusion of the VMV cPPT/cts on the transduction efficiency of the VMV vectors as this element was absent in a VMV vector system described previously¹⁹. However, testing of vectors with 2 potential VMV cPPT/cts elements showed that they had titres similar to those of vectors containing neither element (FIG. 12). This suggests either that the fragments of pol tested do not encode a functional cPPT/cts or that the VMV cPPT does not confer any advantage in the cell lines tested. In either case, inclusion of the cPPT/cts in our VMV vectors does not appear to contribute to the improved infectivity over those previously reported.

A further potential explanation for the greater infectivity of the VMV vector system described here might relate to the specific viral genomes used. Lentiviruses show a high degree of sequence variation both between and within infected individuals and it is therefore possible that the vectors described by Berkowitz were based on a suboptimal viral genome. We believe this is unlikely as those vectors were derived from an infectious molecular clone of VMV (LV1-1KS1⁵²). Here, we based the transfer vector on KV1772, an Icelandic strain of VMV⁵³, whereas the packaging plasmid was derived from EV1, a British strain of the virus⁵⁴. This was done to reduce the sequence similarity between the two plasmids. As it is not straightforward to directly compare titres of vectors prepared in different studies (due to the variety of experimental factors that influence the titre measured^{1,55}), determination of the reason for the functional differences between previous VMV vectors and those described here would require a direct head-to-head comparison of the two systems. This was beyond the scope of the present study.

An important feature of our VMV vectors is the instability of a fraction of marker gene expression (see FIGS. 6 and 8). This does not appear to be a result of pseudotransduction³¹ (FIG. 7) and instead it appears likely that some of the EGFP measured at 3 days post-transduction is expressed from non-integrated viral DNA intermediates that are lost over time. Notably, this fraction of non-stable transduction is also lost in non-dividing cells over a similar time-frame (FIG. 8). Previous studies described the presence of large amounts of unintegrated linear viral DNA in VMV-infected cells^{56,57} and it is possible that transgene expression arises from similar linear forms of VMV vector DNA, although we have not tested this directly. Circular episomal forms of the vector genome are produced in VMV-vector transduced cells (RKM, unpublished data) and it is likely that these are more stable than the linear form, which could explain the persistence of transgene expression in arrested cells.

Despite the instability of transduction, the remaining stable portion of expression still provides titres over 10⁵ TU/mL for unconcentrated VMV vectors and when pseudotyped with the VSV G protein they can be concentrated to at least 10⁷ TU/mL by ultracentrifugation. It is possible that titres can be enhanced by further development of the vector plasmids or production conditions, such as through optimization of the packaging element on the transfer plasmid⁵⁸. Similarly, the use of alternative internal promoters to drive transgene expression has been shown to improve transduction efficiency of EIAV vectors and to enhance the stability of transgene expression⁵⁹. Work is ongoing to assess whether similar modifications can improve the titre of VMV vectors.

The approach taken to increase the biosafety of the VMV lentiviral vector was informed by previous work by various groups using HIV-1 derived lentiviral vectors^{13,32,33}. In the SIN vectors, LTR-mediated transcription was reduced to almost background levels, while inclusion of an internal promoter to drive transgene expression confirmed that the vectors are infectious (FIG. 9). We generated vectors with two inactivating deletions, with and without the TATA box (SIN1 and SIN2 respectively) and they behaved similarly in our assays. Importantly, vector titre and the level of EGFP expression were not compromised.

Analysis of VMV IDLV showed that EGFP expression was more stable in cell cycle arrested cells than in dividing cells (FIG. 8). By analogy with studies on other lentiviral vector systems, it appears likely that this is due to a defect in integration resulting from the mutations introduced into the IN coding region. However, it is notable that 2%-5% of dividing cells transduced with VMV IDLV retained EGFP expression 12 days post-transduction, indicating that some integration may have occurred. Studies on HIV vectors have also shown residual levels of integration with IDLV most likely due to cellular processes involved in recombination and DNA repair acting independently of IN^{35,42}. The degree of background integration varies between studies ranging from 10,000 to 10-fold lower than that obtained with integration-competent vectors and therefore VMV IDLVs appear to be at the high end of this range.

Interestingly, combining the self-inactivating and integration-defective VMV lentiviral vectors resulted in only a 5-fold reduction in transduction efficiency (Table 3). This reduction is consistent with data reported in other lentiviral vector systems⁴¹⁻⁴⁴. Additional experiments are necessary to characterize the SIN and integration-defective VMV vectors more completely; for example, to determine transgene expression levels compared to integration-competent vectors. In addition, modification of the LTR deletion might be beneficial in further reducing the background activity of the SIN LTR. Nevertheless, the studies presented here demonstrate that SIN IDLV can be constructed from VMV and provide a starting point for future work.

The major target cell types for VMV in vivo are macrophages and dendritic cells¹⁸ and here we show that the VMV vectors can efficiently transduce ovine MDDC cultured in vitro. Notably, a significantly higher level of transduction was observed with VMV vectors compared to vectors derived from HIV-1. This is consistent with a previous report⁶⁰ and while it is most likely attributable to ovine-specific blocks to HIV-1 infection, it demonstrates one situation where VMV vectors might be superior to HIV vectors. However, we also discovered that ovine MDDC in culture exhibit an increased rate of apoptosis following transduction with VMV vectors. The reason for this is unclear but it is dependent on reverse transcription of vector RNA to DNA (FIG. 11), indicating that cell death is triggered by cellular sensing of vector DNA or at a subsequent step during vector entry. Studies on HIV-1 have shown that the cellular DNA sensors interferon gamma-inducible factor 16 (IF116) and cyclic GMP-AMP synthase (cGAS) bind HIV-1 cDNA during infection of human cells and activate interferon-responses through STING-TBK2 and IRF3/7⁶¹⁻⁶³. IF116 is also known to trigger inflammasome activation in human CD4⁺ T-cells in response to HIV-1, resulting in pyroptosis⁶⁴. Thus, it appears likely that DNA sensing is responsible for the cytopathic effect induced by VMV vectors in these experiments. Further studies are in progress to examine the mechanisms by which VMV vectors trigger cell death in ovine MDDCs and to determine whether IF116 or cGAS are involved.

The cytopathicity of VMV vectors observed in primary ovine MDDCs suggests that there might be limitations for the use of these vectors for stable gene delivery in vivo. However, one intended future use of the VMV vector system is to assess its suitability as a vaccine delivery system in ruminants and in that situation the induction of inflammatory responses and cell death could potentially be beneficial as long as there was also sufficient expression of vaccine-encoded antigen⁶⁵. In addition, if VMV vectors were to induce apoptosis during vaccine gene delivery in vivo, that could potentially assist vector clearance from the host. Interestingly, the mode of action of alum, an adjuvant commonly used in veterinary vaccines, is to induce apoptosis in the cells it targets and thereby promote immune responses^{66,67}.

In recent years HIV-1 has become the standard lentiviral vector system and the most recently optimized versions have been exploited in a variety of clinical trials^{15-17,68,69} demonstrating that lentiviral vectors are safe and effective. Furthermore, non-HIV vector systems have proved valuable in some instances; for example, EIAV vectors have been employed in clinical gene therapy in Parkinson's disease¹¹ and FIV vectors have been reported to be more efficient than HIV vectors at deriving transgenic cattle⁷⁰ and in delivering genes to porcine airway⁷¹. Thus, it is possible that VMV vectors might find particular application in studies on small ruminants or other livestock species, such as in the generation of transgenic sheep or goats or in experimental gene targeting studies in vivo. In summary, the vectors described here are a valuable starting point for further VMV vector development and provide a novel reporter virus system for studying VMV replication in vitro.

### Example 2

Louping ill virus is a tick-borne flavivirus that causes infection of the brain and spinal cord in a number of species, chiefly in sheep and red grouse. Louping ill virus has an RNA genome that encodes structural proteins (capsid, premembrane and envelope) and several non-structural proteins. The premembrane and envelope proteins can be expressed in recombinant form as a polyprotein that is cleaved into mature proteins by cellular enzymes.

The prME coding region of louping ill virus was subcloned into the pCVW-SIN2 ovine lentiviral vector plasmid (SEQ ID NO:22) under the control of the human cytomegalovirus immediate early promoter. Ovine lentiviral vector particles encoding LIV prME (denoted CVW-LIV-prME) were prepared by transfection and their ability to deliver the gene to target cells tested in vitro by transduction of 293T cells. Immunoblots were performed on cell extracts and on samples of cell culture supernatant, which demonstrated that the prME protein is expressed in transduced cells and also released from cells into the culture supernatant. This confirmed that the CVW-LIV-prME vector is able to function for gene transfer of LIV-prME (FIG. 13).

The CVW-LIV-prME lentiviral vector was then administered to sheep (n−4) by intramuscular injection. The inoculum contained 1×10⁶ transducing units of vector per sheep. Four additional sheep that did not receive the vector were maintained as negative control. Blood samples were taken 18 days before and 0, 10, 24 and 37 days after administration of the vector, at which point a second ‘booster’ dose was given. Control sheep again received no treatment. A further blood sample was taken 10 days after the booster inoculation.

Serum was prepared from each blood sample and used to measure antibodies to LIV using a previously published haemagglutination inhibition assay (HAI)⁸⁰ (Casals and Brown (1954), Journal of Experimental Medicine, 99:429-49). The results indicated that sheep that received the CVW-LIV-prME lentiviral vector had antibodies to LIV whereas the four control sheep did not. The titre of these antibodies initially reduced at each bleed post-first inoculation but showed a marked increase after the booster inoculation (FIG. 14). This provides evidence that the CVW-LIV-prME lentiviral vector elicited a memory immune response to LIV in sheep.

### REFERENCES

1 Sakuma, T., Barry, M. A. & Ikeda, Y. Lentiviral vectors: basic to translational. Biochem. J. 443, 603-618, doi:10.1042/bj20120146 (2012).
2 Dull, T. et al. A third-generation lentivirus vector with a conditional packaging system. J. Virol. 72, 8463-8471 (1998).
3 Naldini, L. et al. In vivo gene delivery and stable transduction of nondividing cells by a lentiviral vector. Science 272, 263-267 (1996).
4 Olsen, J. C. EIAV, CAEV and other lentivirus vector systems. Somat. Cell. Molec. Gen. 26, 131-145 (2001).
5 Schnell, T., Foley, P., Wirth, M., Munch, J. & Uberla, K. Development of a self-inactivating, minimal lentivirus vector based on simian immunodeficiency virus. Hum. Gene. Ther. 11, 439-447, doi:10.1089/10430340050015905 (2000).
6 Mitrophanous, K. et al. Stable gene transfer to the nervous system using a non-primate lentiviral vector. Gene. Ther. 6, 1808-1818, doi:10.1038/sj.gt.3301023 (1999).
7 Olsen, J. C. Gene transfer vectors derived from equine infectious anemia virus. Gene. Ther. 5, 1481-1487 (1998).
8 Poeschla, E. M., Wong-Staal, F. & Looney, D. J. Efficient transduction of nondividing human cells by feline immunodeficiency virus lentiviral vectors. Nat. Med. 4, 354-357 (1998).
9 Berkowitz, R. D. et al. Construction and molecular analysis of gene transfer systems derived from bovine immunodeficiency virus. J. Virol. 75, 3371-3382 (2001).
10 Matukonis, M. et al. Development of second- and third-generation bovine immunodeficiency virus-based gene transfer systems. Hum. Gene. Ther. 13, 1293-1303, doi:10.1089/104303402760128522 (2002).
11 Palfi, S. et al. Long-term safety and tolerability of ProSavin, a lentiviral vector-based gene therapy for Parkinson's disease: a dose escalation, open-label, phase 1/2 trial. Lancet 383, 1138-1146, doi:10.1016/50140-6736(13)61939-X (2014).
12 Miyoshi, H., Blomer, U., Takahashi, M., Gage, F. H. & Verma, I. M. Development of a self-inactivating lentivirus vector. J. Virol. 72, 8150-8157 (1998).
13 Zufferey, R. et al. Self-inactivating lentivirus vector for safe and efficient in vivo gene delivery. J. Virol. 72, 9873-9880 (1998).
14 Leavitt, A. D., Robles, G., Alesandro, N. & Varmus, H. E. Human immunodeficiency virus type 1 integrase mutants retain in vitro integrase activity yet fail to integrate viral DNA efficiently during infection. J. Virol. 70, 721-728 (1996).
15 Cartier, N. et al. Hematopoietic stem cell gene therapy with a lentiviral vector in X-linked adrenoleukodystrophy. Science 326, 818-823, doi:10.1126/science.1171242 (2009).
16 Biffi, A. et al. Lentiviral hematopoietic stem cell gene therapy benefits metachromatic leukodystrophy. Science 341, 1233158, doi:10.1126/science.1233158 (2013).
17 Aiuti, A. et al. Lentiviral hematopoietic stem cell gene therapy in patients with Wiskott-Aldrich syndrome. Science 341, 1233151, doi:10.1126/science.1233151 (2013).
18 Bertoni, G. & Blacklaws, B. in Lentivirus and Macrophages: Molecular and Cellular Interactions (ed M. Desport) 277-306 (Caister Academic Press, 2010).
19 Berkowitz, R. D., lives, H., Plavec, I. & Veres, G. Gene transfer systems derived from visna virus: analysis of virus production and infectivity. Virology 279, 116-129 (2001).
20 Mselli-Lakhal, L. et al. Defective RNA packaging is responsible for low transduction efficiency of CAEV-based vectors. Arch. Virol. 143, 681-695 (1998).
21 Mselli-Lakhal, L., Guiguen, F., Greenland, T., Mornex, J. F. & Chebloune, Y. Gene transfer system derived from the caprine arthritis-encephalitis lentivirus. J. Virol. Methods 136, 177-184, doi:10.1016/j.jviromet.2006.05.006 (2006).
22 Mátrai, J., Chuah, M. K. L. & VandenDriessche, T. Recent advances in lentiviral vector development and applications. Mol. Ther. 18, 477-490 (2010).
23 Bjarnadottir, H., Gudmundsson, B., Gudnason, J. & Jonsson, J. J. Encapsidation determinants located downstream of the major splice donor in the maedi-visna virus leader region. J. Virol. 80, 11743-11755, doi:10.1128/jvi.01284-06 (2006).
24 Tiley, L. S. & Cullen, B. R. Structural and functional analysis of the visna virus Rev-response element. J. Virol. 66, 3609-3615 (1992).
25 Zufferey, R., Donello, J. E., Trono, D. & Hope, T. J. Woodchuck hepatitis virus posttranscriptional regulatory element enhances expression of transgenes delivered by retroviral vectors. J. Virol. 73, 2886-2892 (1999).
26 Logan, A. C. et al. Factors influencing the titer and infectivity of lentiviral vectors. Hum. Gene Ther. 15, 976-988, doi:10.1089/hum.2004.15.976 (2004).
27 Bray, M. et al. A small element from the Mason-Pfizer monkey virus genome makes human immunodeficiency virus type 1 expression and replication Rev-independent. Proc. Natl. Acad. Sci. USA 91, 1256-1260 (1994).
28 Naldini, L., Blamer, U., Gage, F. H., Trono, D. & Verma, I. M. Efficient transfer, integration, and sustained long-term expression of the transgene in adult rat brains injected with a lentiviral vector. Proc. Natl. Acad. Sci. USA 93, 11382-11388 (1996).
29 Liu, M. L., Winther, B. L. & Kay, M. A. Pseudotransduction of hepatocytes by using concentrated pseudotyped vesicular stomatitis virus G glycoprotein (VSV-G)-Moloney murine leukemia virus-derived retrovirus vectors: comparison of VSV-G and amphotropic vectors for hepatic gene transfer. J. Virol. 70, 2497-2502 (1996).
30 Pichlmair, A. et al. Tubulovesicular structures within vesicular stomatitis virus G protein-pseudotyped lentiviral vector preparations carry DNA and stimulate antiviral responses via Toll-like receptor 9. J. Virol. 81, 539-547 (2007).
31 Haas, D. L., Case, S. S., Crooks, G. M. & Kohn, D. B. Critical factors influencing stable transduction of human CD34(+) cells with HIV-1-derived lentiviral vectors. Mol. Ther. 2, 71-80, doi:10.1006/mthe.2000.0094 (2000).
32 Wanisch, K. & Yáñez-Munoz, R. J. Integration-deficient lentiviral vectors: a slow coming of age. Mol. Ther. 17, 1316-1332 (2009).
33 Miyoshi, H., Blamer, U., Takahashi, M., Gage, F. H. & Verna, I. M. Development of a self-inactivating lentivirus vector. J Virol 72, 8150-8157 (1998).
34 Störmann, K. D., Schlecht, M. C. & Pfaff, E. Comparative studies of bacterially expressed integrase proteins of caprine arthritis-encephalitis virus, maedi-visna virus and human immunodeficiency virus type 1. J. Gen. Virol. 76, 1651-1663, doi:10.1099/0022-1317-76-7-1651 (1995).
35 Gaur, M. & Leavitt, A. D. Mutations in the human immunodeficiency virus type 1 integrase D,D(35)E motif do not eliminate provirus formation. J. Virol. 72, 4678-4685 (1998).
36 Basu, V. P. et al. Strand transfer events during HIV-1 reverse transcription. Virus Res. 134, 19-38 (2008).
37 Yu, S. F. et al. Self-inactivating retroviral vectors designed for transfer of whole genes into mammalian cells. Proc. Natl. Acad. Sci. USA 83, 3194-3198 (1986).
38 Gabuzda, D. H., Hess, J. L., Small, J. A. & Clements, J. E. Regulation of the visna virus long terminal repeat in macrophages involves cellular factors that bind sequences containing AP-1 sites. Mol. Cell. Biol. 9, 2728-2733 (1989).
39 Hess, J. L., Small, J. A. & Clements, J. E. Sequences in the visna virus long terminal repeat that control transcriptional activity and respond to viral trans-activation: involvement of AP-1 sites in basal activity and trans-activation. J. Virol. 63, 3001-3015 (1989).
40 Sutton, K. A., Lin, C. T., Harkiss, G. D., McConnell, I. & Sargan, D. Regulation of the long terminal repeat in visna virus by a transcription factor related to the AML/PEBP2/CBF superfamily. Virology 229, 240-250 (1997).
41 Apolonia, L. et al. Stable gene transfer to muscle using non-integrating lentiviral vectors. Mol. Ther. 15, 1947-1954, doi:10.1038/sj.mt.6300281 (2007).
42 Banasik, M. B. & McCray, P. B. Integrase-defective lentiviral vectors: progress and applications. Gene Ther. 17, 150-157, doi:10.1038/gt.2009.135 (2010).
43 Philippe, S. et al. Lentiviral vectors with a defective integrase allow efficient and sustained transgene expression in vitro and in vivo. Proc. Natl. Acad. Sci. USA 103, 17684-17689, doi:10.1073/pnas.0606197103 (2006).
44 Vargas, J. J., Gusella, G. L., Najfeld, V., Klotman, M. E. & Cara, A. Novel integrase-defective lentiviral episomal vectors for gene transfer. Hum. Gene. Ther. 15, 361-372, doi:10.1089/104303404322959515 (2004).
45 Charneau, P. & Clavel, F. A single-stranded gap in human immunodeficiency virus unintegrated linear DNA defined by a central copy of the polypurine tract. J. Virol. 65, 2415-2421 (1991).
46 Charneau, P. et al. HIV-1 reverse transcription a termination step at the centre of the genome. J. Mol. Biol. 241, 651-662 (1994).
47 Stetor, S. R. et al. Characterization of (+) strand initiation and termination sequences located at the center of the equine infectious anemia virus genome. Biochemistry 38, 3656-3667, doi:10.1021/bi982764I (1999).
48 Whitwam, T., Peretz, M. & Poeschla, E. Identification of a central DNA flap in feline immunodeficiency virus. J. Virol. 75, 9407-9414, doi:10.1128/jvi.75.19.9407-9414.2001 (2001).
49 Harris, J. D. et al. Visna virus DNA: discovery of a novel gapped structure. Virology 113, 573-583 (1981).
50 Sonigo, P. et al. Nucleotide sequence of the visna lentivirus: relationship to the AIDs virus. Cell 42, 369-382 (1985).
51 Metharom, P. et al. Novel bovine lentiviral vectors based on Jembrana disease virus. J. Gene Med. 2, 176-185 (2000).
52 Staskus, K. A. et al. Isolation of replication-competent molecular clones of visna virus. Virology 181, 228-240 (1991).
53 Andresson, O. S. et al. Nucleotide sequence and biological properties of a pathogenic proviral molecular clone of neurovirulent visna virus. Virology 193, 89-105 (1993).
54 Sargan, D. et al. Nucleotide sequence of EV1, a British isolate of maedi-visna virus. J. Gen. Virol. 72, 1893-1903 (1991).
55 Loewen, N. et al. in Methods in Molecular Biology: Lentivirus Gene Engineering Protcols Vol. 229 (ed M. Frederico) 251-271 (Humana Press 2003).
56 Harris, J. D. et al. Slow virus visna: reproduction in vitro of virus from extrachromosomal DNA. Proc. Natl. Acad. Sci. USA 81, 7212-7215 (1984).
57 List, J. & Haase, A. T. Integration of visna virus DNA occurs and may be necessary for productive infection. Virology 237, 189-197, doi:10.1006/viro.1997.8785 (1997).
58 Kemler, I., Barraza, R. & Poeschla, E. M. Mapping the encapsidation determinants of feline immunodeficiency virus. J. Virol. 76, 11889-11903 (2002).
59 O'Rourke, J. P., Olsen, J. C. & Bunnell, B. A. Optimization of equine infectious anemia derived vectors for hematopoietic cell lineage gene transfer. Gene Ther. 12, 22-29, doi:10.1038/sj.gt.3302350 (2005).
60 Karponi, G., Kritas, S., Petridou, E. & Papanikolaou, E. Efficient Transduction and Expansion of Ovine Macrophages for Gene Therapy Implementations. Vet Sci 5, doi:10.3390/vetsci5020057 (2018).
61 Herzner, A. M. et al. Sequence-specific activation of the DNA sensor cGAS by Y-form DNA structures as found in primary HIV-1 cDNA. Nat. Immunol. 16, 1025-1033, doi:10.1038/ni.3267 (2015).
62 Gao, D. et al. Cyclic GMP-AMP synthase is an innate immune sensor of HIV and other retroviruses. Science 341, 903-906, doi:10.1126/science.1240933 (2013).
63 Jakobsen, M. R. et al. IFI16 senses DNA forms of the lentiviral replication cycle and controls HIV-1 replication. Proc. Natl. Acad. Sci. USA 110, E4571-4580, doi:10.1073/pnas.1311669110 (2013).
64 Monroe, K. M. et al. IF116 DNA sensor is required for death of lymphoid CD4 T cells abortively infected with HIV. Science 343, 428-432, doi:10.1126/science.1243640 (2014).
65 Jakobsen, M. R., Olagnier, D. & Hiscott, J. Innate immune sensing of HIV-1 infection. Curr. Opin. HIV AIDS 10, 96-102, doi:10.1097/coh.0000000000000129 (2015).
66 Kool, M. et al. Cutting edge: alum adjuvant stimulates inflammatory dendritic cells through activation of the NALP3 inflammasome. J. Immunol. 181, 3755-3759 (2008).
67 Jivani, H. M. et al. Veterinary vaccines: past, present and future—a review. Int. J. Sci. Env. Tech. 5, 3473-3485 (2016).
68 Kaiser, J. Gene therapy. Beta-thalassemia treatment succeeds, with a caveat. Science 326, 1468-1469, doi:10.1126/science.326.5959.1468-b (2009).
69 Cavazzana-Calvo, M. et al. Transfusion independence and HMGA2 activation after gene therapy of human beta-thalassaemia. Nature 467, 318-322, doi:10.1038/nature09328 (2010).
70 Xu, Y. N. et al. Production of transgenic Korean native cattle expressing enhanced green fluorescent protein using a FIV-based lentiviral vector injected into MII oocytes. J Genet Genomics 40, 37-43, doi:10.1016/j.jgg.2012.11.001 (2013).
71 Sinn, P. L. et al. Lentiviral vector gene transfer to porcine airways. Mol. Ther. Nucleic Acids 1, e56, doi:10.1038/mtna.2012.47 (2012).
72 Ikeda, Y. et al. Continuous high-titer HIV-1 vector production. Nat. Biotechnol. 21, 569-572, doi:10.1038/nbt815 (2003).
73 Arnaud, F. et al. Interplay between ovine bone marrow stromal cell antigen 2/tetherin and endogenous retroviruses. J. Virol. 84, 4415-4425, doi:10.1128/jvi.00029-10 (2010).
74 Crandell, R. A., Fabricant, C. G. & Nelson-Rees, W. A. Development, characterization, and viral susceptibility of a feline (Felis catus) renal cell line (CRFK). In vitro 9, 176-185 (1973).
75 Giard, D. J. et al. In vitro cultivation of human tumors: establishment of cell lines derived from a series of solid tumors. J. Natl. Cancer Inst. 51, 1417-1423 (1973).
76 Madin, S. H. & Darby, N. B., Jr. Established kidney cell lines of normal adult bovine and ovine origin. Proc. Soc. Exp. Biol. Med. 98, 574-576 (1958).
77 Da Silva Teixeira, M. F. et al. Immortalization of caprine fibroblasts permissive for replication of small ruminant lentiviruses. Am. J. Vet. Res. 58, 579-584 (1997).
78 Stabel, J. R. & Stabel, T. J. Immortalization and characterization of bovine peritoneal macrophages transfected with SV40 plasmid DNA. Vet. Immunol. Immunopathol. 45, 211-220 (1995).
79 Grigorov, B., Rabilloud, J., Lawrence, P. & Gerlier, D. Rapid titration of measles and other viruses: optimization with determination of replication cycle length. PLoS One 6, e24135, doi:10.1371/journal.pone.0024135 (2011).
80 Casals and Brown (1954), Journal of Experimental Medicine, 99:429-49

TABLE 1
Antibodies and isotype controls used for flow cytometry

| | | | | | |
|---|---|---|---|---|---|
| | | | | | Catalogue |
| Antibody | Clone | Isotype | Dilution | Supplier | Code |

Isotype Controls

| | | | | | |
|---|---|---|---|---|---|
| Mouse IgG1 isotype^{a} | VPM21 | IgG1 | 1:500 | In house^{e} | — |
| Mouse IgG2a isotype^{c} | VPM20 | IgG2a | 1:5 | In house | — |
| Mouse IgG2b isotype^{a} | VPM22 | IgG2b | 1:500 | In house | — |
| Rat IgG2b isotype | eB149/10H5 | IgG2b | 1:500 | eBioscience | 12-4031-81 |

Primary mAb

| | | | | | |
|---|---|---|---|---|---|
| Mouse anti-ovine CD14^{b} | VPM65 | IgG1 | 1:1000 | In house | — |
| Rat anti-ovine MHC class II^{c} | SW73.2 | IgG2b | 1:500 | In house | — |
| Mouse anti-bovine CD80 | IL-A159 | IgG1 | 1:500 | Bio-Rad | MCA2436F |
| Mouse anti-bovine CD40 | IL-A156 | IgG1 | 1:500 | Bio-Rad | MCA2431GA |
| Mouse anti-bovine CD11b | CC132 | IgG2b | 1:500 | Bio-Rad | MCA1425GA |
| Mouse anti-bovine CD1w2 | CC20 | IgG2a | 1:500 | Bio-Rad | MCA2058G |
| Mouse anti Human CD163 | EDHu-1 | IgG1 | 1:50 | Bio-Rad | MCA1853 |
| Mouse anti-bovine CD172a | ILA24 | IgG1 | Neat | In house | — |

Secondary pAb

| | | | | | |
|---|---|---|---|---|---|
| Goat anti-rat IgG(H + L): RPE | — | — | 1:500 | Bio-Rad | 3030-09 |
| Goat anti-mouse IgG(H + L): | — | — | 1:1000 | ThermoFisher | A-11029 |
| AlexaFluor 488 | | | | | |

TABLE 2
Percentage EGFP-positive cells following transduction with integration-competent and integration-defective
VMV lentiviral vectors from day 2 to day 12.

| | | Day 2 | Day 4 | Day 6 | Day 8 | Day 10 | Day 12 |
|---|---|---|---|---|---|---|---|
| | | Average | Average | Average | Average | Average | Average |
| | | (%) | (%) | (%) | (%) | (%) | (%) |
| | Vector Construct | Min, Max | Min, Max | Min, Max | Min, Max | Min, Max | Min, Max |
| Dividing | CVW-CG | 73.3 | 53.7 | 33.9 | 32.8 | 29.6 | 24.5 |
| | | 64.2, 80.2 | 46.6, 59.5 | 22.6, 52.7 | 19.6, 55.4 | 18.5, 48.7 | 18.6, 35.2 |
| | CVW-CG/ΔIN1 | 70.9 | 45.9 | 28.5 | 14.7 | 3.3 ** | 2.8 *** |
| | | 52.3, 82.6 | 33.6, 57.5 | 17.1, 36.0 | 4.1, 38.6 | 1.1, 6.5 | 1.9, 5.4 |
| | CVW-CG/ΔIN2 | 71.0 | 49.7 | 32.2 | 23.1 | 5.1 ** | 3.4 *** |
| | | 49.6, 82.4 | 41.5, 60.1 | 20.3, 51.4 | 11.2, 42.8 | 3.4, 6.9 | 2.6, 4.6 |
| Non- | CVW-CG | 73.3 | 68.8 | 43.8 | 41.7 | 44.6 | 40.4 |
| Dividing | | 64.2, 80.2 | 60.9, 78.2 | 34.6, 64.7 | 31.8, 55.6 | 34.7, 61.1 | 29.6, 56.7 |
| | CVW-CG/ΔIN1 | 70.9 | 64.6 | 41.8 | 42.1 | 43.8 | 36.2 |
| | | 52.3, 82.6 | 52.3, 81.5 | 30.4, 64.7 | 29.7, 65.2 | 31.0, 69.1 | 27.3, 51.4 |
| | CVW-CG/ΔIN2 | 71.0 | 60.4 | 43.4 | 47.1 | 45.3 | 35.0 |
| | | 49.6, 82.4 | 48.5, 83.2 | 35.2, 61.7 | 35.3, 64.2 | 36.5, 61.9 | 31.1, 45.6 |

TABLE 3
Infectivity of CVW-CG and SIN/ΔIN VMV lentiviral vectors.

| | Percentage of EGFP- | |
|---|---|---|
| | positive CRFK cells* | Fold differences in |
| Vector | Actual (Converted) | infectivity |
| CVW-CG | 43.5 (57.23) | 1.00 |
| CVW-SIN1-CG/ΔIN1 | 12.53 | −4.57 |
| CVW-SIN1-CG/ΔIN2 | 14.74 | −3.88 |
| CVW-SIN2-CG/ΔIN1 | 11.89 | −4.81 |
| CVW-SIN2-CG/ΔIN2 | 11.98 | −4.78 |

*Input vector volumes were standardized by RT activity. The converted value for CVW-CG indicates the ‘corrected’ MOI to avoid under-estimation of titre due to the non-linearity of transduced cells relative to infectious titre at high MOI.

1. A plasmid comprising a nucleic acid sequence encoding a promoter, a nucleic acid sequence encoding an encapsidation element, a nucleic acid sequence encoding a rev-responsive element (RRE), a site for the insertion of a nucleic acid for transfer, a woodchuck hepatitis virus post-transcriptional regulatory element (WPRE), and at least a portion of a 5′ terminal repeat and at least a portion of a 3′ terminal repeat;
wherein the nucleic acid sequence encoding an encapsidation element and the nucleic acid sequence encoding at least a portion of a 5′ terminal repeat and at least a portion of a 3′ terminal repeat are small ruminant lentivirus nucleic acid sequences.

2. The plasmid of claim 1, wherein the WPRE comprises the sequence of SEQ ID NO: 1, or a fragment or variant thereof.

3. The plasmid of claim 1, wherein the nucleic acid sequence encoding a promoter comprises SEQ ID NO: 2, or a fragment or variant thereof.

4. The plasmid of claim 1, wherein at least a portion of the 5′ long terminal repeat comprises at least one of SEQ ID NO:3, SEQ ID NO:4, SEQ ID NO:5 and/or SEQ ID NO:6, or a fragment or variant thereof.

5. The plasmid of claim 1, wherein at least a portion of the 3′ terminal repeat comprises at least one of SEQ ID NO:3, SEQ ID NO:4, SEQ ID NO:5, SEQ ID NO:6 and/or SEQ ID NO:7, or a fragment or variant thereof.

6. The plasmid of claim 1, wherein the nucleic acid sequence encoding an encapsidation element comprises SEQ ID NO: 10 and/or SEQ ID NO: 13, or a fragment or variant thereof.

7. The plasmid of claim 1, wherein the nucleic acid sequence encoding an RRE comprises SEQ ID NO: 14, or a fragment or variant thereof.

8. The plasmid of claim 1, wherein the plasmid is part of a lentiviral vector system, wherein the lentiviral vector system optionally comprises a packaging plasmid comprising a nucleic acid sequence encoding a gag polyprotein and a gag-pol polyprotein.

9. The lentiviral vector system of claim 8, wherein the nucleic acid sequence encoding a gag polyprotein and a gal-pol polyprotein comprises SEQ ID NO:24, or a fragment or variant thereof.

10. The lentiviral vector system according to claim 8 further comprising an additional plasmid comprising a nucleic acid sequence encoding an envelope protein.

11. The lentiviral vector system of claim 10, wherein the nucleic acid sequence encoding an envelope protein is derived from a vesicular stomatitis virus (VSG).

12. The lentiviral vector system according to claim 8, the system further comprising an additional plasmid comprising a nucleic acid sequence encoding a rev protein.

13. The lentiviral vector system of claim 12, wherein the nucleic acid sequence encoding the rev protein is a small ruminant lentivirus nucleic acid sequence

14. A lentiviral vector particle derived from a small ruminant lentivirus, wherein the lentiviral vector particle comprises a nucleic acid sequence encoding an encapsidation element, a nucleic acid sequence encoding a rev-responsive element (RRE), a nucleic acid sequence for transfer, a woodchuck hepatitis virus post-transcriptional regulatory element (WPRE), and at least a portion of a 5′ terminal repeat and at least a portion of a 3′ terminal repeat;
wherein the nucleic acid sequence encoding an encapsidation element and the nucleic acid sequence encoding at least a portion of a 5′ terminal repeat and at least a portion of a 3′ terminal repeat are small ruminant lentivirus nucleic acid sequences.

15.-16. (canceled)

17. A method of transducing a cell, said method comprising contacting a cell to be transduced with a lentiviral vector particle derived from a small ruminant lentivirus according to claim 14.

18. A method of raising an immune response in an animal, said method comprising administering an immunogenic amount of a lentiviral vector particle derived from a small ruminant lentivirus according to claim 14 to an animal, optionally wherein the animal is an ovine animal, a caprine animal, an equine animal, a porcine animal, a bovine animal or a human.

19. (canceled)

20. An immunogenic composition or vaccine comprising the lentiviral vector particle according to claim 14.

21. The immunogenic composition or vaccine according to claim 20, wherein the immunogenic composition or vaccine further comprises pharmaceutically acceptable and/or sterile excipients, carriers and/or diluents.

22. The immunogenic composition or vaccine according to claim 20, wherein the immunogenic composition or vaccine further comprises or is admixed with an antigen, a polypeptide and/or an adjuvant.

23. The plasmid according to claim 1, wherein the site for the insertion of the nucleic acid for transfer is adjacent to the WPRE.

24. The plasmid according to claim 1, wherein at least a portion of the 5′ long terminal repeat is between the nucleic acid sequence encoding a promoter and the site for the insertion of a nucleic acid for transfer.

25. The plasmid according t claim 1, wherein the portion of the 3′ long terminal repeat does not contain a TATA box nucleic acid sequence.

26. The plasmid according to claim 1, wherein a nucleic acid for transfer is inserted into the site for the insertion of a nucleic acid for transfer.

27. (canceled)

28. The lentiviral vector system according to claim 8, wherein the packaging plasmid comprises a nucleic acid sequence encoding one or more Mason Pfizer monkey virus constitutive transport elements (CTE) downstream of the nucleic acid sequence encoding the gag and gag-pol polyproteins.

29. (canceled)


SEQUENCE LISTING (53 sequences)

SEQ ID NO: 1 - DNA, 596 bp, Woodchuck hepatitis B virus
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcg

SEQ ID NO: 2 - DNA, 593 bp, Human cytomegalovirus
agttattaaagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctag

SEQ ID NO: 3 - DNA, 13 bp, Visna virus
cgcttgctagctg

SEQ ID NO: 4 - DNA, 254 bp, Visna virus
actgtcaggacagagaacaaatgcctaccctggaaaatgactatgtagagttataggaaggtcatgtcactgttaccagaaatcatagtcaggatgacacagcaaatgtaaccggttaccagaaatcatagtcaggatgacacagcaaatgtaaccgcaagttctgcttttttgcgctgagtcatgtagcagctgatggttaagtcataaccgcagatgtaaacaagttgcctatataagccgcttgctagctg

SEQ ID NO: 5 - DNA, 98 bp, Visna virus
gggaaaagcagagtgctttggagagctcgaaggaaagagtctccgggcctctcctgcctgcctgaaaagctcaataaaggagttggctgatatctgag

SEQ ID NO: 6 - DNA, 63 bp, Visna virus
cttgcctggttattatcgggattcgttactaattccgtgcaacaccggagcggatctcgcagc

SEQ ID NO: 7 - DNA, 104 bp, Visna virus
gacacaactggaggacaatgccctatataaccctgctacccatattggtgatatggcaatggatggaagagaatggatggaatggagagaatcagcacaaaaag

SEQ ID NO: 8 - DNA, 114 bp, Visna virus
actgtcaggacagagaacaaatgcctaccctggaaaatgactatgtagagttataggaaggtcatgtcacttaattaagtaaacaagttgcctatataagccgcttgctagctg

SEQ ID NO: 9 - DNA, 88 bp, Visna virus
actgtcaggacagagaacaaatgcctaccctggaaaatgactatgtagagttataggaaggtcatgtcacttaattaattgctagctg

SEQ ID NO: 10 - DNA, 327 bp, Visna virus
tggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagac

SEQ ID NO: 11 - DNA, 16 bp, Visna virus
aaaaaagaaagggtgg

SEQ ID NO: 12 - DNA, 5227 bp, Artificial Sequence
  Sequence derived from a small ruminant lentivirus
gcggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaagcatggttcgatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcgaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcactgttaccagaaatcatagtcaggatgacacagcaaatgtaaccggttaccagaaatcatagtcaggatgacacagcaaatgtaaccgcaagttctgcttttttgcgctgagtcatgtagcagctgatggttaagtcataaccgcagatgtaaacaagttgcctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgac

SEQ ID NO: 13 - DNA, 713 bp, Visna virus
taggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtgga

SEQ ID NO: 14 - DNA, 202 bp, Visna virus
gggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctag

SEQ ID NO: 15 - DNA, 529 bp, Visna virus
gttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagcttt

SEQ ID NO: 16 - DNA, 765 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaagcatggttcgatctga

SEQ ID NO: 17 - DNA, 6247 bp, Artificial Sequence
  plasmid
cggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcataggcccctttgggaaattgctgactgattcaaggtccaattcaggacttacagatagaaccttcctagatctaggtgttccctattcgaagcttgctacgatttaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcactgttaccagaaatcatagtcaggatgacacagcaaatgtaaccggttaccagaaatcatagtcaggatgacacagcaaatgtaaccgcaagttctgcttttttgcgctgagtcatgtagcagctgatggttaagtcataaccgcagatgtaaacaagttgcctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgg

SEQ ID NO: 18 - DNA, 6846 bp, Artificial Sequence
  plasmid
gcggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcataggcccctttgggaaattgctgactgattcaaggtccaattcaggacttacagatagaaccttcctagatctcgagctcaagcttcgaagcatggttcgatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcgaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcactgttaccagaaatcatagtcaggatgacacagcaaatgtaaccggttaccagaaatcatagtcaggatgacacagcaaatgtaaccgcaagttctgcttttttgcgctgagtcatgtagcagctgatggttaagtcataaccgcagatgtaaacaagttgcctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgg

SEQ ID NO: 19 - DNA, 8117 bp, Artificial Sequence
  plasmid
cggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaagcatggttcgatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcgaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcactgttaccagaaatcatagtcaggatgacacagcaaatgtaaccggttaccagaaatcatagtcaggatgacacagcaaatgtaaccgcaagttctgcttttttgcgctgagtcatgtagcagctgatggttaagtcataaccgcagatgtaaacaagttgcctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgg

SEQ ID NO: 20 - DNA, 2905 bp, Artificial Sequence
  pBluescript plasmid
gtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgggcggccgc

SEQ ID NO: 21 - DNA, 6706 bp, Artificial Sequence
  plasmid
gcggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcataggcccctttgggaaattgctgactgattcaaggtccaattcaggacttacagatagaaccttcctagatctcgagctcaagcttcgaagcatggttcgatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcgaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcacttaattaagtaaacaagttgcctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgg

SEQ ID NO: 22 - DNA, 6679 bp, Artificial Sequence
  plasmid
cggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagccgcttgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagctggcgcccaacgtggggctcgacaaagaatcagaagaaaaatgagagttacgggaccacggacgctgctcctgtgaggacggcgaggagagtaacggacacggacaaaaagtgaaagaaagcttcggggacgcctgaagtaaggtaagagagacacctactggggaagtagggaatagcccttcagtgaaagagaaagtgttgcttgggcacaggaggagggttcgcgaccccttaaaagacggaggggcacgggcgtcctcctgggccgaggggacacaagagcaacactggtaaggaagccgccgtggtgaggctagctagagactaggcgaagcaaggctcaaaggagaaaaagggataccccgagctcaaggaagtaattaaagcaacttgtaaaataagggtagggcccgggaaggagaccttgacagaagggaattgtctatgggcattaaaaactatagactttatatttgaggatttaaaaacagagccgtggacgattacaaaaatgtatacagtatgggatagattaaaagggctaactccggaggaaacaagcaaaagagaattcgcctccttgcaagctacgttggcttgcataatgtgtagtcaaatgggcatgaagcccgagacagtgcaggcagcaaagggaataataagtatgaaagaaggactacacgaaaataaggaggccaagggggagaaggtagagcaactctaccccaacttagagaaacatagggaagtttatcctattgtgaatttgcaagcaggagggagaagttggaaggcggtagagtcagtagtcttccagcaactgcaaacagtggcaatgcagcatggacttgtgtccgaggattttgagaggcaattggcatattatgctactacctggactagtaaagatatattagaagtattggctatgatgcctgggaatagagcacagaaggaattaatacaaggaaaattaaatgaagaagcagaaaggtgggtaagacaaaatccacccgggccgaatgtcctcacggtggaatattagtcagtcatcgatgggataggcttggttattgtgctagccatcatggcaataatcgctgctgcaggagctggtctcggtgtcgcaaacgccgtgcagcaatcctataccaggacggctgtccagtctcttgctaacgcaactgctgcccagcaggaagtgttagaagcatcgtatgccatggtacagcatatagccaaaggaataagaatcctagcatgcagtgtgcgttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatccggcttacagccggcaccccggctggggaagccaagtaagaaatgttgaactgtgataagcctgtctaggatgcataggcccctttgggaaattgctgactgattcaaggtccaattcaggacttacagatagaaccttcctagatctcgagctcaagcttcgaagcatggttcgatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtcgaaatacctagcctcgagggttctttgaaatagcgctttgacacaactggaggacaatgccctatataaccctgctacccatattggtgatatggcaatggatggaagagaatggatggaatggagagaatcagcacaaaaagaaaaaagaaagggtggactgtcaggacagagaacaaatgcctaccctggaaaatgactatgtagagttataggaaggtcatgtcacttaattaattgctagctggggaaaagcagagtgctttggagagctcgaaggaaagagtctccgggcctctcctgcctgcctgaaaagctcaataaaggagttggctgatatctgagcttgcctggttattatcgggattcgttactaattccgtgcaacaccggagcggatctcgcagcgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtgg

SEQ ID NO: 23 - DNA, 419 bp, Mason-Pfizer monkey virus
caagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgt

SEQ ID NO: 24 - DNA, 4541 bp, Visna virus
atggcgaagcaaggctcaagggagaaaaagggataccccgagctcaaagaggtaataaggagaacatgtaggataagagtagggcccgggaaggagaccttgacagaagggaactgtctatgggcattaaaaactgtagactttatatttgaagatttaaaagaagagccgtggacccttacaaaaatgtatacagtatgggatagattaaaacagttaactccagaagagacaagtaaaagagagtttgcctccttgcaggccacaatggcttgccttatgtgtagtcagctgggtatgaaacccgagacagtgcaagcagcaaggggaataatgcatatgaaagaaggactacaggagaataaggaggaaaaggagaaaaaggtagaacaactctaccctaatttagagaaacatagagaagtgtaccctattgtaaatctgcaagcaggggggagaagttggaaggcggtagattcagtagtcttccagcaattgcaaactgtggctatgcagcatggccttgtgtccgaggattttgaaagacagctggcgtattatgctactacatggacaagcaaggatatattagaagtattggccatgatgcctgggaacagagcacagaaagagctgattcagggaaaattaaatgaagaagcagaaagatgggtgaggcagaacccgccagggccaaatgtcctcacggtggatcaaatcatgggagtaggacaaacaaatcaacaggcatcacaggctaatatggatcaagcaaggcaactgtgcttgcagtgggtcataacagccttgagagcggtaaggcatatgtcgcatagaccaggtaacccaatgctggtaaagcagaagaatactgagagttttgaagatttcatagcaagattgctggaagcaattgatgcagaaccagtcacagatcctataaaaacatatttaaaagtgactctgtcgtacacgaatgctagtacagattgtcaaaagcaaatggacagagttttgggaactagggtacaacaggcaacagtagaagaaaaaatgcaagcatgtcgagatgtaggttcagagggatttaaaatgcagctgttagcgcaggctttaagaccacaacggagagagggaaatggagggccaagtcaaaaatgttataattgtggaaaacaaggacatcttgcaagacaatgcaggcaagggataatttgccatcagtgtggaaaaagaggacatatgagaaaagactgtcggcaaaagaagaaggataacatacagctgcagggaaacaacaggagggggccacgtgtggtgccgtccgcgcctcctatgttgtaacagaagcaccaccaaaggcagaaataaaggtagggacaacatggagaatgttattagtagacaccggagcagataggacaatagtaagatatcatgataattcgggaataccaaaaggaagaataaaattgcagggtatagggggaattatagaaggagaaaaatgggacaaagtggcgttacagtataaagaaaaaagaatcttgggtaccatagtagtactgcctagcagtccagtggaggtgttgggaagggataatatgggagaattaggaataggactaattatggcaaatctggaagaaaggaaaatccctattaccaaggtaagcctaaaagaaggctgcaagggacctcatatagcgcagtggcctttgactcaagaaaaacttgaaggattaaaagaaatagtggaaagattagaaaaagaagggaaattaggtagggcacctccgcattggacatgtaacactcctatattttgcattaaaaagaaatcaggaaaatggagaatgttaatagacttcagggaattgaataaacaaacagaagatttggcagaggcacagttggggttaccacatccgggaggattgcagaaaaagaaacatgtaacagtattggatataggagatgcatattttacaatacctttgtatgaaccttatagacagtatacatgttttaccatgctgagtcccaataatttgggaccttgtgtaaggtattattggaaggtgctgccacaaggatggaagttgagtccctcagtgtatcaatttaccatgcaggagatattaagagattggataagggaacaccctatggtgcaatttgggatatatatggatgatatctatataggcagtgatttagaaatgggggaacacagaagaatagtagaagaacttgccagttatattgcccaatatgggtttatgctgccggaagagaagaggcaagaaggatatccagcaaattggcttggatttgaactacatccagagagatggaagtttcaaaaacataagcttccagatatggaagaaggaccaataacgttaaataaattgcagaaattagtaggagaattagtttggaggcaatcattgatagggaaaagtataccaaatatactgaaattgatggaaggagatagagcgttacaaagtgtaaggaatgtagagaaaatacatatagaagaatgggaaggatgtaaaagaaaactagaagaaatggaagggaattattataatgcagaaaaagatgtttatggacaagtagattggggaaataaagcaatagaatatatagtgttccaagaaaaagggaaaccattatgggtgaatgtagtacatagcattaagaatttgagccaagcacagcagatcattaaagcggcacaaaagcttacacaagaagtaataataagaacaggaaaaataccatggatactactgccaggaaaagaggaggactggatcttggaactgcaggtgggaaatatcacgtggatgccatcattttggtcatgttataggggatcagtaagatggaaaaagagaaatgtagtaacagaagtagtagaggggccaacatattatacagatggagggaagaaaaatggagaaggaagcttaggatatattgcttccaccggggaaaaatgtagaatgcatgagaaagggacaaatcaacaactagaattaagggcaattgaggaagcatgtaaacagggaccaagcaaaatgaatatagtaacagatagtagatatgcatttgagttcataataaggaactgggatgaagaagttataaggaatccaatacaggcacgaatcatgaagttaatacatagtaaggagaaggtagggatacattgggtgccaggccataaagggattcctcaaaatgaagaaatagataaatatatttcagaagtatttttagcaaaagaaggaaatgggatagtaaagaaaagagcagaggatgctgggtatgatttgatatgcccacaagaggtaagtatcccagcaggacaagtaaagaagattccaattgatttaagaataaatttaagaaaaaatcaatgggctatgatagggacaaaaagcagttttgcaagtaagggagtatttgtacaaggaggaatagtagattcaggatatcagggaatcatacaagtagtaatttataacagcaatgacgaagaggtcattataccccaggggaggaaatttgcacagttaattctcatgccgctgatacatgaggaattagagccatggggggaaacaagaaaaacagaaagaggaaatcagggatttggatcaacaggagcatattggattgaaaatattcccttggcggaagaagatcatagtaaatggcatcaagatgctcaatcattgcatctagaatttgacataccaagaacagcggctgaagatatagtgcaacaatgtgaaatatgtcaagaaaataaaatgcctaatacaatgagaggaagtaacaagaggggaatagatcattggcaagtggattacactcattttgaagataagatattactagtatgggtagaaacaaattcgggattaatttatgcagaaagggtgaaaggggagacaggacaagaatttagagtaacagctatgaagtggtatgctctgtttgccccaaaatcattgcaatctgataatgggccagcatttgtagcagaagcaacacaactgctaatgaaatatttagggataatacatacaacagggataccttggaatccacagtctcaagctctagtggaaagggctcatcagactttaaagaaaacaattgaaaaacttgttcctatgttctctgcatttgaatcagctgtcgcagctgcattaatagctctaaatataaaaagaaagggtgggctagggacaagccctatggatatattcatatttaataaggaacagcaaagaatacaacaacagtataaattaaatcaagaaaaaattcgattttgttattacagaatcagaaaaagaggacacccaggcgactggctgggaccgtctcaggtactctgggaaggggaaggagcaatagtcgtaaaagatagaactctagataagtatttagtaatagctaacaaagatgttaaattcataccgcaaccaaaagaaatacaaaaagagcaaaaatag

SEQ ID NO: 25 - DNA, 27 bp, Artificial Sequence
  primer
ggcaagtggattacactcattttgaag

SEQ ID NO: 26 - DNA, 27 bp, Artificial Sequence
  primer
cctggccactagagcttgagactgtgg

SEQ ID NO: 27 - DNA, 26 bp, Artificial Sequence
  primer
gagtgtaatccacttgccaatgatct

SEQ ID NO: 28 - DNA, 27 bp, Artificial Sequence
  primer
caagctctagtggccagggctcatcag

SEQ ID NO: 29 - DNA, 27 bp, Artificial Sequence
  primer
ggcaagtggcctacactcattttgaag

SEQ ID NO: 30 - DNA, 26 bp, Artificial Sequence
  primer
gagtgtaggccacttgccaatgatct

SEQ ID NO: 31 - DNA, 517 bp, Human cytomegalovirus
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 32 - DNA, 517 bp, Artificial Sequence; identical to SEQ ID NO: 31
  CAG promoter

SEQ ID NO: 33 - DNA, 1010 bp, Artificial Sequence
  chimeric intro
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagc

SEQ ID NO: 34 - DNA, 10 bp, Artificial Sequence
  kozak sequence
gccaccatgg

SEQ ID NO: 35 - DNA, 4343 bp, Artificial Sequence
  plasmid
gcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 36 - DNA, 11706 bp, Artificial Sequence
  plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcacgcgtggtacctctagagtcgacagtgccaccatggcgaagcaaggctcaagggagaaaaagggataccccgagctcaaagaggtaataaggagaacatgtaggataagagtagggcccgggaaggagaccttgacagaagggaactgtctatgggcattaaaaactgtagactttatatttgaagatttaaaagaagagccgtggacccttacaaaaatgtatacagtatgggatagattaaaacagttaactccagaagagacaagtaaaagagagtttgcctccttgcaggccacaatggcttgccttatgtgtagtcagctgggtatgaaacccgagacagtgcaagcagcaaggggaataatgcatatgaaagaaggactacaggagaataaggaggaaaaggagaaaaaggtagaacaactctaccctaatttagagaaacatagagaagtgtaccctattgtaaatctgcaagcaggggggagaagttggaaggcggtagattcagtagtcttccagcaattgcaaactgtggctatgcagcatggccttgtgtccgaggattttgaaagacagctggcgtattatgctactacatggacaagcaaggatatattagaagtattggccatgatgcctgggaacagagcacagaaagagctgattcagggaaaattaaatgaagaagcagaaagatgggtgaggcagaacccgccagggccaaatgtcctcacggtggatcaaatcatgggagtaggacaaacaaatcaacaggcatcacaggctaatatggatcaagcaaggcaactgtgcttgcagtgggtcataacagccttgagagcggtaaggcatatgtcgcatagaccaggtaacccaatgctggtaaagcagaagaatactgagagttttgaagatttcatagcaagattgctggaagcaattgatgcagaaccagtcacagatcctataaaaacatatttaaaagtgactctgtcgtacacgaatgctagtacagattgtcaaaagcaaatggacagagttttgggaactagggtacaacaggcaacagtagaagaaaaaatgcaagcatgtcgagatgtaggttcagagggatttaaaatgcagctgttagcgcaggctttaagaccacaacggagagagggaaatggagggccaagtcaaaaatgttataattgtggaaaacaaggacatcttgcaagacaatgcaggcaagggataatttgccatcagtgtggaaaaagaggacatatgagaaaagactgtcggcaaaagaagaaggataacatacagctgcagggaaacaacaggagggggccacgtgtggtgccgtccgcgcctcctatgttgtaacagaagcaccaccaaaggcagaaataaaggtagggacaacatggagaatgttattagtagacaccggagcagataggacaatagtaagatatcatgataattcgggaataccaaaaggaagaataaaattgcagggtatagggggaattatagaaggagaaaaatgggacaaagtggcgttacagtataaagaaaaaagaatcttgggtaccatagtagtactgcctagcagtccagtggaggtgttgggaagggataatatgggagaattaggaataggactaattatggcaaatctggaagaaaggaaaatccctattaccaaggtaagcctaaaagaaggctgcaagggacctcatatagcgcagtggcctttgactcaagaaaaacttgaaggattaaaagaaatagtggaaagattagaaaaagaagggaaattaggtagggcacctccgcattggacatgtaacactcctatattttgcattaaaaagaaatcaggaaaatggagaatgttaatagacttcagggaattgaataaacaaacagaagatttggcagaggcacagttggggttaccacatccgggaggattgcagaaaaagaaacatgtaacagtattggatataggagatgcatattttacaatacctttgtatgaaccttatagacagtatacatgttttaccatgctgagtcccaataatttgggaccttgtgtaaggtattattggaaggtgctgccacaaggatggaagttgagtccctcagtgtatcaatttaccatgcaggagatattaagagattggataagggaacaccctatggtgcaatttgggatatatatggatgatatctatataggcagtgatttagaaatgggggaacacagaagaatagtagaagaacttgccagttatattgcccaatatgggtttatgctgccggaagagaagaggcaagaaggatatccagcaaattggcttggatttgaactacatccagagagatggaagtttcaaaaacataagcttccagatatggaagaaggaccaataacgttaaataaattgcagaaattagtaggagaattagtttggaggcaatcattgatagggaaaagtataccaaatatactgaaattgatggaaggagatagagcgttacaaagtgtaaggaatgtagagaaaatacatatagaagaatgggaaggatgtaaaagaaaactagaagaaatggaagggaattattataatgcagaaaaagatgtttatggacaagtagattggggaaataaagcaatagaatatatagtgttccaagaaaaagggaaaccattatgggtgaatgtagtacatagcattaagaatttgagccaagcacagcagatcattaaagcggcacaaaagcttacacaagaagtaataataagaacaggaaaaataccatggatactactgccaggaaaagaggaggactggatcttggaactgcaggtgggaaatatcacgtggatgccatcattttggtcatgttataggggatcagtaagatggaaaaagagaaatgtagtaacagaagtagtagaggggccaacatattatacagatggagggaagaaaaatggagaaggaagcttaggatatattgcttccaccggggaaaaatgtagaatgcatgagaaagggacaaatcaacaactagaattaagggcaattgaggaagcatgtaaacagggaccaagcaaaatgaatatagtaacagatagtagatatgcatttgagttcataataaggaactgggatgaagaagttataaggaatccaatacaggcacgaatcatgaagttaatacatagtaaggagaaggtagggatacattgggtgccaggccataaagggattcctcaaaatgaagaaatagataaatatatttcagaagtatttttagcaaaagaaggaaatgggatagtaaagaaaagagcagaggatgctgggtatgatttgatatgcccacaagaggtaagtatcccagcaggacaagtaaagaagattccaattgatttaagaataaatttaagaaaaaatcaatgggctatgatagggacaaaaagcagttttgcaagtaagggagtatttgtacaaggaggaatagtagattcaggatatcagggaatcatacaagtagtaatttataacagcaatgacgaagaggtcattataccccaggggaggaaatttgcacagttaattctcatgccgctgatacatgaggaattagagccatggggggaaacaagaaaaacagaaagaggaaatcagggatttggatcaacaggagcatattggattgaaaatattcccttggcggaagaagatcatagtaaatggcatcaagatgctcaatcattgcatctagaatttgacataccaagaacagcggctgaagatatagtgcaacaatgtgaaatatgtcaagaaaataaaatgcctaatacaatgagaggaagtaacaagaggggaatagatcattggcaagtggattacactcattttgaagataagatattactagtatgggtagaaacaaattcgggattaatttatgcagaaagggtgaaaggggagacaggacaagaatttagagtaacagctatgaagtggtatgctctgtttgccccaaaatcattgcaatctgataatgggccagcatttgtagcagaagcaacacaactgctaatgaaatatttagggataatacatacaacagggataccttggaatccacagtctcaagctctagtggaaagggctcatcagactttaaagaaaacaattgaaaaacttgttcctatgttctctgcatttgaatcagctgtcgcagctgcattaatagctctaaatataaaaagaaagggtgggctagggacaagccctatggatatattcatatttaataaggaacagcaaagaatacaacaacagtataaattaaatcaagaaaaaattcgattttgttattacagaatcagaaaaagaggacacccaggcgactggctgggaccgtctcaggtactctgggaaggggaaggagcaatagtcgtaaaagatagaactctagataagtatttagtaatagctaacaaagatgttaaattcataccgcaaccaaaagaaatacaaaaagagcaaaaatagggagatagggcctcggttgccgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 37 - DNA, 11706 bp, Artificial Sequence
  plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcacgcgtggtacctctagagtcgacagtgccaccatggcgaagcaaggctcaagggagaaaaagggataccccgagctcaaagaggtaataaggagaacatgtaggataagagtagggcccgggaaggagaccttgacagaagggaactgtctatgggcattaaaaactgtagactttatatttgaagatttaaaagaagagccgtggacccttacaaaaatgtatacagtatgggatagattaaaacagttaactccagaagagacaagtaaaagagagtttgcctccttgcaggccacaatggcttgccttatgtgtagtcagctgggtatgaaacccgagacagtgcaagcagcaaggggaataatgcatatgaaagaaggactacaggagaataaggaggaaaaggagaaaaaggtagaacaactctaccctaatttagagaaacatagagaagtgtaccctattgtaaatctgcaagcaggggggagaagttggaaggcggtagattcagtagtcttccagcaattgcaaactgtggctatgcagcatggccttgtgtccgaggattttgaaagacagctggcgtattatgctactacatggacaagcaaggatatattagaagtattggccatgatgcctgggaacagagcacagaaagagctgattcagggaaaattaaatgaagaagcagaaagatgggtgaggcagaacccgccagggccaaatgtcctcacggtggatcaaatcatgggagtaggacaaacaaatcaacaggcatcacaggctaatatggatcaagcaaggcaactgtgcttgcagtgggtcataacagccttgagagcggtaaggcatatgtcgcatagaccaggtaacccaatgctggtaaagcagaagaatactgagagttttgaagatttcatagcaagattgctggaagcaattgatgcagaaccagtcacagatcctataaaaacatatttaaaagtgactctgtcgtacacgaatgctagtacagattgtcaaaagcaaatggacagagttttgggaactagggtacaacaggcaacagtagaagaaaaaatgcaagcatgtcgagatgtaggttcagagggatttaaaatgcagctgttagcgcaggctttaagaccacaacggagagagggaaatggagggccaagtcaaaaatgttataattgtggaaaacaaggacatcttgcaagacaatgcaggcaagggataatttgccatcagtgtggaaaaagaggacatatgagaaaagactgtcggcaaaagaagaaggataacatacagctgcagggaaacaacaggagggggccacgtgtggtgccgtccgcgcctcctatgttgtaacagaagcaccaccaaaggcagaaataaaggtagggacaacatggagaatgttattagtagacaccggagcagataggacaatagtaagatatcatgataattcgggaataccaaaaggaagaataaaattgcagggtatagggggaattatagaaggagaaaaatgggacaaagtggcgttacagtataaagaaaaaagaatcttgggtaccatagtagtactgcctagcagtccagtggaggtgttgggaagggataatatgggagaattaggaataggactaattatggcaaatctggaagaaaggaaaatccctattaccaaggtaagcctaaaagaaggctgcaagggacctcatatagcgcagtggcctttgactcaagaaaaacttgaaggattaaaagaaatagtggaaagattagaaaaagaagggaaattaggtagggcacctccgcattggacatgtaacactcctatattttgcattaaaaagaaatcaggaaaatggagaatgttaatagacttcagggaattgaataaacaaacagaagatttggcagaggcacagttggggttaccacatccgggaggattgcagaaaaagaaacatgtaacagtattggatataggagatgcatattttacaatacctttgtatgaaccttatagacagtatacatgttttaccatgctgagtcccaataatttgggaccttgtgtaaggtattattggaaggtgctgccacaaggatggaagttgagtccctcagtgtatcaatttaccatgcaggagatattaagagattggataagggaacaccctatggtgcaatttgggatatatatggatgatatctatataggcagtgatttagaaatgggggaacacagaagaatagtagaagaacttgccagttatattgcccaatatgggtttatgctgccggaagagaagaggcaagaaggatatccagcaaattggcttggatttgaactacatccagagagatggaagtttcaaaaacataagcttccagatatggaagaaggaccaataacgttaaataaattgcagaaattagtaggagaattagtttggaggcaatcattgatagggaaaagtataccaaatatactgaaattgatggaaggagatagagcgttacaaagtgtaaggaatgtagagaaaatacatatagaagaatgggaaggatgtaaaagaaaactagaagaaatggaagggaattattataatgcagaaaaagatgtttatggacaagtagattggggaaataaagcaatagaatatatagtgttccaagaaaaagggaaaccattatgggtgaatgtagtacatagcattaagaatttgagccaagcacagcagatcattaaagcggcacaaaagcttacacaagaagtaataataagaacaggaaaaataccatggatactactgccaggaaaagaggaggactggatcttggaactgcaggtgggaaatatcacgtggatgccatcattttggtcatgttataggggatcagtaagatggaaaaagagaaatgtagtaacagaagtagtagaggggccaacatattatacagatggagggaagaaaaatggagaaggaagcttaggatatattgcttccaccggggaaaaatgtagaatgcatgagaaagggacaaatcaacaactagaattaagggcaattgaggaagcatgtaaacagggaccaagcaaaatgaatatagtaacagatagtagatatgcatttgagttcataataaggaactgggatgaagaagttataaggaatccaatacaggcacgaatcatgaagttaatacatagtaaggagaaggtagggatacattgggtgccaggccataaagggattcctcaaaatgaagaaatagataaatatatttcagaagtatttttagcaaaagaaggaaatgggatagtaaagaaaagagcagaggatgctgggtatgatttgatatgcccacaagaggtaagtatcccagcaggacaagtaaagaagattccaattgatttaagaataaatttaagaaaaaatcaatgggctatgatagggacaaaaagcagttttgcaagtaagggagtatttgtacaaggaggaatagtagattcaggatatcagggaatcatacaagtagtaatttataacagcaatgacgaagaggtcattataccccaggggaggaaatttgcacagttaattctcatgccgctgatacatgaggaattagagccatggggggaaacaagaaaaacagaaagaggaaatcagggatttggatcaacaggagcatattggattgaaaatattcccttggcggaagaagatcatagtaaatggcatcaagatgctcaatcattgcatctagaatttgacataccaagaacagcggctgaagatatagtgcaacaatgtgaaatatgtcaagaaaataaaatgcctaatacaatgagaggaagtaacaagaggggaatagatcattggcaagtggattacactcattttgaagataagatattactagtatgggtagaaacaaattcgggattaatttatgcagaaagggtgaaaggggagacaggacaagaatttagagtaacagctatgaagtggtatgctctgtttgccccaaaatcattgcaatctgataatgggccagcatttgtagcagaagcaacacaactgctaatgaaatatttagggataatacatacaacagggataccttggaatccacagtctcaagctctagtggccagggctcatcagactttaaagaaaacaattgaaaaacttgttcctatgttctctgcatttgaatcagctgtcgcagctgcattaatagctctaaatataaaaagaaagggtgggctagggacaagccctatggatatattcatatttaataaggaacagcaaagaatacaacaacagtataaattaaatcaagaaaaaattcgattttgttattacagaatcagaaaaagaggacacccaggcgactggctgggaccgtctcaggtactctgggaaggggaaggagcaatagtcgtaaaagatagaactctagataagtatttagtaatagctaacaaagatgttaaattcataccgcaaccaaaagaaatacaaaaagagcaaaaatagggagatagggcctcggttgccgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 38 - DNA, 11706 bp, Artificial Sequence
  plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcacgcgtggtacctctagagtcgacagtgccaccatggcgaagcaaggctcaagggagaaaaagggataccccgagctcaaagaggtaataaggagaacatgtaggataagagtagggcccgggaaggagaccttgacagaagggaactgtctatgggcattaaaaactgtagactttatatttgaagatttaaaagaagagccgtggacccttacaaaaatgtatacagtatgggatagattaaaacagttaactccagaagagacaagtaaaagagagtttgcctccttgcaggccacaatggcttgccttatgtgtagtcagctgggtatgaaacccgagacagtgcaagcagcaaggggaataatgcatatgaaagaaggactacaggagaataaggaggaaaaggagaaaaaggtagaacaactctaccctaatttagagaaacatagagaagtgtaccctattgtaaatctgcaagcaggggggagaagttggaaggcggtagattcagtagtcttccagcaattgcaaactgtggctatgcagcatggccttgtgtccgaggattttgaaagacagctggcgtattatgctactacatggacaagcaaggatatattagaagtattggccatgatgcctgggaacagagcacagaaagagctgattcagggaaaattaaatgaagaagcagaaagatgggtgaggcagaacccgccagggccaaatgtcctcacggtggatcaaatcatgggagtaggacaaacaaatcaacaggcatcacaggctaatatggatcaagcaaggcaactgtgcttgcagtgggtcataacagccttgagagcggtaaggcatatgtcgcatagaccaggtaacccaatgctggtaaagcagaagaatactgagagttttgaagatttcatagcaagattgctggaagcaattgatgcagaaccagtcacagatcctataaaaacatatttaaaagtgactctgtcgtacacgaatgctagtacagattgtcaaaagcaaatggacagagttttgggaactagggtacaacaggcaacagtagaagaaaaaatgcaagcatgtcgagatgtaggttcagagggatttaaaatgcagctgttagcgcaggctttaagaccacaacggagagagggaaatggagggccaagtcaaaaatgttataattgtggaaaacaaggacatcttgcaagacaatgcaggcaagggataatttgccatcagtgtggaaaaagaggacatatgagaaaagactgtcggcaaaagaagaaggataacatacagctgcagggaaacaacaggagggggccacgtgtggtgccgtccgcgcctcctatgttgtaacagaagcaccaccaaaggcagaaataaaggtagggacaacatggagaatgttattagtagacaccggagcagataggacaatagtaagatatcatgataattcgggaataccaaaaggaagaataaaattgcagggtatagggggaattatagaaggagaaaaatgggacaaagtggcgttacagtataaagaaaaaagaatcttgggtaccatagtagtactgcctagcagtccagtggaggtgttgggaagggataatatgggagaattaggaataggactaattatggcaaatctggaagaaaggaaaatccctattaccaaggtaagcctaaaagaaggctgcaagggacctcatatagcgcagtggcctttgactcaagaaaaacttgaaggattaaaagaaatagtggaaagattagaaaaagaagggaaattaggtagggcacctccgcattggacatgtaacactcctatattttgcattaaaaagaaatcaggaaaatggagaatgttaatagacttcagggaattgaataaacaaacagaagatttggcagaggcacagttggggttaccacatccgggaggattgcagaaaaagaaacatgtaacagtattggatataggagatgcatattttacaatacctttgtatgaaccttatagacagtatacatgttttaccatgctgagtcccaataatttgggaccttgtgtaaggtattattggaaggtgctgccacaaggatggaagttgagtccctcagtgtatcaatttaccatgcaggagatattaagagattggataagggaacaccctatggtgcaatttgggatatatatggatgatatctatataggcagtgatttagaaatgggggaacacagaagaatagtagaagaacttgccagttatattgcccaatatgggtttatgctgccggaagagaagaggcaagaaggatatccagcaaattggcttggatttgaactacatccagagagatggaagtttcaaaaacataagcttccagatatggaagaaggaccaataacgttaaataaattgcagaaattagtaggagaattagtttggaggcaatcattgatagggaaaagtataccaaatatactgaaattgatggaaggagatagagcgttacaaagtgtaaggaatgtagagaaaatacatatagaagaatgggaaggatgtaaaagaaaactagaagaaatggaagggaattattataatgcagaaaaagatgtttatggacaagtagattggggaaataaagcaatagaatatatagtgttccaagaaaaagggaaaccattatgggtgaatgtagtacatagcattaagaatttgagccaagcacagcagatcattaaagcggcacaaaagcttacacaagaagtaataataagaacaggaaaaataccatggatactactgccaggaaaagaggaggactggatcttggaactgcaggtgggaaatatcacgtggatgccatcattttggtcatgttataggggatcagtaagatggaaaaagagaaatgtagtaacagaagtagtagaggggccaacatattatacagatggagggaagaaaaatggagaaggaagcttaggatatattgcttccaccggggaaaaatgtagaatgcatgagaaagggacaaatcaacaactagaattaagggcaattgaggaagcatgtaaacagggaccaagcaaaatgaatatagtaacagatagtagatatgcatttgagttcataataaggaactgggatgaagaagttataaggaatccaatacaggcacgaatcatgaagttaatacatagtaaggagaaggtagggatacattgggtgccaggccataaagggattcctcaaaatgaagaaatagataaatatatttcagaagtatttttagcaaaagaaggaaatgggatagtaaagaaaagagcagaggatgctgggtatgatttgatatgcccacaagaggtaagtatcccagcaggacaagtaaagaagattccaattgatttaagaataaatttaagaaaaaatcaatgggctatgatagggacaaaaagcagttttgcaagtaagggagtatttgtacaaggaggaatagtagattcaggatatcagggaatcatacaagtagtaatttataacagcaatgacgaagaggtcattataccccaggggaggaaatttgcacagttaattctcatgccgctgatacatgaggaattagagccatggggggaaacaagaaaaacagaaagaggaaatcagggatttggatcaacaggagcatattggattgaaaatattcccttggcggaagaagatcatagtaaatggcatcaagatgctcaatcattgcatctagaatttgacataccaagaacagcggctgaagatatagtgcaacaatgtgaaatatgtcaagaaaataaaatgcctaatacaatgagaggaagtaacaagaggggaatagatcattggcaagtggcctacactcattttgaagataagatattactagtatgggtagaaacaaattcgggattaatttatgcagaaagggtgaaaggggagacaggacaagaatttagagtaacagctatgaagtggtatgctctgtttgccccaaaatcattgcaatctgataatgggccagcatttgtagcagaagcaacacaactgctaatgaaatatttagggataatacatacaacagggataccttggaatccacagtctcaagctctagtggccagggctcatcagactttaaagaaaacaattgaaaaacttgttcctatgttctctgcatttgaatcagctgtcgcagctgcattaatagctctaaatataaaaagaaagggtgggctagggacaagccctatggatatattcatatttaataaggaacagcaaagaatacaacaacagtataaattaaatcaagaaaaaattcgattttgttattacagaatcagaaaaagaggacacccaggcgactggctgggaccgtctcaggtactctgggaaggggaaggagcaatagtcgtaaaagatagaactctagataagtatttagtaatagctaacaaagatgttaaattcataccgcaaccaaaagaaatacaaaaagagcaaaaatagggagatagggcctcggttgccgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcgggaattcgattcaagcttatgacctttattaaacatcaaattgagagcatccaggccaaacctatacaagtccattatcatcgccttgaacaagaagacagtggtggctcatatttgaccttaacataggccacctcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgtcttttgttttttataattaaaaagggtgacatgtccggagccgtgctgcccggatgatgtcttggcctctgtttgctctagctccatgttatgaatttaagatggcgtatttcctggttcttctccgtaatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 39 - DNA, 4456 bp, Artificial Sequence
  plasmid
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgtcgccaccatggcaagcaaggagtccaaaccgagccgaaccactcgtagagggatggagccacctttacgcgaaacctggaatcaggtcttgcaggagcttgtgaaacgccaacagcaggaagaggaagagcaacagggtcttgtgtctggcctacaagcgtctaaggccgatcagatctacaccggcaattccggggatcggtcaacaggagggattggaggcaaaacgaagaagaaacggggatggtacaaatggctcagaaagctgagagctagggaaaagaacatacccagtcagttctatccagacatggagagcaacatggttggcatggagaacctgacactggagactcagctggaggacaatgccctctacaatcccgccacacacatcggtgacatggctatggatgggagggaatggatggaatggcgggaatcagcacagaaggagaaacgcaaaggtggcctgagtggacagaggaccaacgcctatcctggcaagtgataaagggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 40 - DNA, 27 bp, Artificial Sequence
  primer
ttaagtgacatgaccttcctataactc

SEQ ID NO: 41 - DNA, 27 bp, Artificial Sequence
  primer
ttaagtaaacaagttgcctatataagc

SEQ ID NO: 42 - DNA, 541 bp, Visna virus
gttaacgttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaaaagaaaaccaagggtatgagtattgggaaacatcaggaaaacagtggaaaatggagataagacgagatttggatctgatagcccaaataaattttagaaattgatggcaatataaaagccagggagaatggaaaacaataggggtctggtatgaatcaccaggggattacaagggaaaagagaatcagttttggttccattggagaatagctctctgcagctgtaacaaaacaaggtgggatatacgggaattcatgatagggaagcataggtgggatttatgtaaatcgtgtatacaaggggagatagttaagaatacaaatccaagaagcttacaacgcttagctttggatcc

SEQ ID NO: 43 - DNA, 512 bp, Visna virus
gttaacattagaaaaacttatacctatgtttaacgcgtttgaatcagccctcgcagggaccctcattactctaaatataaaaagaaagggtgggctagggacaagccctatggatatatttatatttaataaggaacaacaaagaatacagcaacaaagtaaatcaaaacaagaaaaaattcgattttgttattacagaacaagaaaaagagggcatccaggagagtggcaaggaccaacacaggtactttggggcggggacggtgcgattgtagtgaaagacagaggcacagatagatatctggtgatagctaacaaagtagttaagttcataccgccaccaaaagaaatacaaaaagaataaagcaagggagataggaccccagttgccactatgggcatggaaagaaacagcatttagtataaatcaggaaccctattggtatagtaccataaggctacaagggttgatgtggaataaaagagggcataaacttatgtttgtaggatcc

SEQ ID NO: 44 - DNA, 47 bp, Artificial Sequence
  primer
gatcgatcgtcgacagtgccaccatggcgaagcaaggctcaarrgag

SEQ ID NO: 45 - DNA, 40 bp, Artificial Sequence
  primer
gatcgatcgcggccgcggcaaccgaggccctatctcccta

SEQ ID NO: 46 - protein, 447 aa, Visna virus
MAKQGSREKKGYPELKEVIRRTCRIRVGPGKETLTEGNCLWALKTVDFIFEDLKEEPWTLTKMYTVWDRLKQLTPEETSKREFASLQATMACLMCSQLGMKPETVQAARGIMHMKEGLQENKEEKEKKVEQLYPNLEKHREVYPIVNLQAGGRSWKAVDSVVFQQLQTVAMQHGLVSEDFERQLAYYATTWTSKDILEVLAMMPGNRAQKELIQGKLNEEAERWVRQNPPGPNVLTVDQIMGVGQTNQQASQANMDQARQLCLQWVITALRAVRHMSHRPGNPMLVKQKNTESFEDFIARLLEAIDAEPVTDPIKTYLKVTLSYTNASTDCQKQMDRVLGTRVQQATVEEKMQACRDVGSEGFKMQLLAQALRPQRREGNGGPSQKCYNCGKQGHLARQCRQGIICHQCGKRGHMRKDCRQKKKDNIQLQGNNRRGPRVVPSAPPML

SEQ ID NO: 47 - protein, 1087 aa, Visna virus
HTAAGKQQEGATCGAVRASYVVTEAPPKAEIKVGTTWRMLLVDTGADRTIVRYHDNSGIPKGRIKLQGIGGIIEGEKWDKVALQYKEKRILGTIVVLPSSPVEVLGRDNMGELGIGLIMANLEERKIPITKVSLKEGCKGPHIAQWPLTQEKLEGLKEIVERLEKEGKLGRAPPHWTCNTPIFCIKKKSGKWRMLIDFRELNKQTEDLAEAQLGLPHPGGLQKKKHVTVLDIGDAYFTIPLYEPYRQYTCFTMLSPNNLGPCVRYYWKVLPQGWKLSPSVYQFTMQEILRDWIREHPMVQFGIYMDDIYIGSDLEMGEHRRIVEELASYIAQYGFMLPEEKRQEGYPANWLGFELHPERWKFQKHKLPDMEEGPITLNKLQKLVGELVWRQSLIGKSIPNILKLMEGDRALQSVRNVEKIHIEEWEGCKRKLEEMEGNYYNAEKDVYGQVDWGNKAIEYIVFQEKGKPLWVNVVHSIKNLSQAQQIIKAAQKLTQEVIIRTGKIPWILLPGKEEDWILELQVGNITWMPSFWSCYRGSVRWKKRNVVTEVVEGPTYYTDGGKKNGEGSLGYIASTGEKCRMHEKGTNQQLELRAIEEACKQGPSKMNIVTDSRYAFEFIIRNWDEEVIRNPIQARIMKLIHSKEKVGIHWVPGHKGIPQNEEIDKYISEVFLAKEGNGIVKKRAEDAGYDLICPQEVSIPAGQVKKIPIDLRINLRKNQWAMIGTKSSFASKGVFVQGGIVDSGYQGIIQVVIYNSNDEEVIIPQGRKFAQLILMPLIHEELEPWGETRKTERGNQGFGSTGAYWIENIPLAEEDHSKWHQDAQSLHLEFDIPRTAAEDIVQQCEICQENKMPNTMRGSNKRGIDHWQVDYTHFEDKILLVWVETNSGLIYAERVKGETGQEFRVTAMKWYALFAPKSLQSDNGPAFVAEATQLLMKYLGIIHTTGIPWNPQSQALVERAHQTLKKTIEKLVPMFSAFESAVAAALIALNIKRKGGLGTSPMDIFIFNKEQQRIQQQYKLNQEKIRFCYYRIRKRGHPGDWLGPSQVLWEGEGAIVVKDRTLDKYLVIANKDVKFIPQPKEIQKEQK

SEQ ID NO: 48 - DNA, 30 bp, Artificial Sequence
  primer
gggatagctgctgccgctatctatataggc

SEQ ID NO: 49 - DNA, 34 bp, Artificial Sequence
  primer
ctatatagatagcggcagcagctatcccaaattg

SEQ ID NO: 50 - DNA, 27 bp, Artificial Sequence
  primer
caaggacatcttgcaagacaatgcagg

SEQ ID NO: 51 - DNA, 31 bp, Artificial Sequence
  primer
cggtggaagcaatatatcctaagcttccttc

SEQ ID NO: 52 - DNA, 504 bp, Visna virus
atggcaagcaaggagtccaaaccgagccgaaccactcgtagagggatggagccacctttacgcgaaacctggaatcaggtcttgcaggagcttgtgaaacgccaacagcaggaagaggaagagcaacagggtcttgtgtctggcctacaagcgtctaaggccgatcagatctacaccggcaattccggggatcggtcaacaggagggattggaggcaaaacgaagaagaaacggggatggtacaaatggctcagaaagctgagagctagggaaaagaacatacccagtcagttctatccagacatggagagcaacatggttggcatggagaacctgacactggagactcagctggaggacaatgccctctacaatcccgccacacacatcggtgacatggctatggatgggagggaatggatggaatggcgggaatcagcacagaaggagaaacgcaaaggtggcctgagtggacagaggaccaacgcctatcctggcaagtga

SEQ ID NO: 53 - protein, 167 aa, Visna virus
MASKESKPSRTTRRGMEPPLRETWNQVLQELVKRQQQEEEEQQGLVSGLQASKADQIYTGNSGDRSTGGIGGKTKKKRGWYKWLRKLRAREKNIPSQFYPDMESNMVGMENLTLETQLEDNALYNPATHIGDMAMDGREWMEWRESAQKEKRKGGLSGQRTNAYPGK